(19) United States
(12) Patent Application Publication
(10) Pub. No.: US 2021/0125681 A1
(43) Pub. Date: Apr. 29, 2021

(54) Method of Modeling Strands Coupled to Rotational Strain

(71) Applicant: Dr. Christine Marie Kennefick, Reston, VA (US)

(72) Inventor: Christine Marie Kennefick, Reston, VA (US)

(21) Appl. No.: 17/064,066

(22) Filed: Oct. 6, 2020

Related U.S. Application Data
(60) Provisional application No. 62/925,371, filed on Oct. 24, 2019.

(51) Int. Cl. G16B 5/00; G16B 15/00; G06F 30/17; G06F 30/23

(52) U.S. Cl. CPC G16B 5/00; G16B 15/00; G06F 2111/10; G06F 30/23; G06F 30/17

(57) ABSTRACT

The method comprises a series a programs that enable a user to model any one or more of numerical data, atoms, electrons, nucleons and atomic clusters as strands, knots and waves that interact with rotational strain. The programs allow placement of strands with varying length scales and shapes at centers, vertices and faces of three-dimensional elements that can have any number of symmetries, internal angles, dimensions and orientations. Parameters in the functions that trace out the strands can be altered by rotational strain components, which arise from rotation of the three-dimensional elements within a shearable medium. For each strand, a program can track its path, extent and number of loops. An advantage of the method is that is allows mapping or model that responds to intricate changes in internal angles and orientation of three dimensional elements, and to intricate changes in functions that trace out the strands.

### FIELD OF THE INVENTION

The present invention relates to methods that model and map data, materials and structures at a microscopic level in the fields of information technology, physics, mechanics, chemistry, and mathematics. In particular, the present invention places knots and strands at centers, vertices and faces of 3D elements with varying symmetry. It then couples shape and movement of strands with elastic strain that the method computes at and near faces of the 3D element after a rotation within a shearable medium.

### BACKGROUND OF THE INVENTION

Computer modeling before experimentation in laboratory can potentially reduce time and cost to develop new micro structures needed in the fields of medicine, sustainable energy, and structural integrity. Methods to model structures, however, can be limited to atomic processes, stress and strain at a continuum level for load bearing components, or can rely on meshes that limit computation in and around grains and atomic clusters with intricate shape and symmetry. Other modeling methods take data as input from an existing structure to display images, data, and graphs, but do not encompass input of new atomic clusters and grains to predict new structures and performance. More broadly, and even further, computer modeling should provide a mapping to track, pinpoint, and predict sources of normal, anomalous, and critical performance.

The present invention seeks to fill a need for modeling, mapping, and predicting structures at a microscopic length scale. The method allows a user to set up three dimensional elements with planar faces that have a size, symmetry, orientation and spacing specified by a user. The present invention goes further in that a user can then selectively place knots and strands at centers, vertices and faces of the three-dimensional elements. The method can then sample the shape and direction of the strands as they interact with each other. The strands can also interact with contact stresses caused by rotation of the three-dimensional elements. With a user's placement of customized three-dimensional elements, along with computation of rotational strain and tracking of strands, the method seeks to model microstructures with both common and irregular internal angles.

### BRIEF SUMMARY OF THE INVENTION

The present invention is a method of steps to be performed by a processor that samples coordinates and tracks shapes of strands for both known and new performance of micro structures and larger structures. The method first collects data for size, symmetry, orientation, spacing and interaction for three dimensional elements with planar faces that can span and encompass atoms, atomic clusters, or grains. The method supports bundled and tapered elements, and, if desired, pyramidal endcaps on the three-dimensional elements.

The method collects as input a length scale and location of strands and knots in and on said three dimensional elements. In the present invention, a strand is a collection of sequential points computed by functions that contain cosine and sine functions. To specify a shape of a strand, the method takes as input the number of cosine and sine functions within a full function that depicts the shape of each strand. The method also takes as input an amplitude, frequency, and phase in each of said cosine and sine functions. A shape of strand can represent a cloudlike electron, or can be smaller and represent an atomic nucleus. Still other functions that represent strands can depict an atomic nucleus with an electronic cloud or bond within a single knot. A strand can also be a simple wave passing through a structure. Parameters within the cosine and sine functions for each strand can be further modified by strain from contact stresses that in turn arise from rotation of said three dimensional elements within a shearable medium. The method can allow a frequency within a cosine or sine function to be dependent upon rotational strain only at a particular point, or upon both rotational strain and a frequency value at a previous sampling point.

The method can track extents, number of loops, number of inflection points and a path of a strand in three orthogonal directions. It can track pairwise interaction between two strands based upon their path or density of inflection points. It can also work with patterns of elastic stress from rotation of the three-dimensional elements without any strands present in a model. One or more strands can also be waves. It is one object of the present invention to set up and model structures with customized internal angles among its micro elements. It is a second object to model desired performance with knots and strands that, at will, can focus upon a single electron or atom, or extend through a structure. It is yet a third object of the invention to provide a method that can map data onto variables and parameters of three-dimensional elements and strands. The goal of this third object is to derive and store information about the original input data.

### BRIEF DESCRIPTION OF THE DRAWINGS

Reference will be made to the accompanying drawings, whose steps and features can span length scales from atomic to continuum levels.

FIG. 1 is a representation of two strands that interact with each other and with rotational strain from three dimensional elements that rotate.

FIG. 2 shows a strand interacting with strain computed in a coordinate system that has its origin on a face of a three-dimensional element and that has its η₁ direction perpendicular to the axis of rotation of said element.

FIG. 3 shows that strands can be located at centers, vertices and faces of both rotating and nonrotating elements.

FIG. 4 is an overview of basic steps in the method and some example applications.

FIG. 5 shows steps to intake commonality and differences in size, symmetry, and arrangement of three-dimensional elements as a whole.

FIG. 6 shows a sequence of steps that sets up the symmetry, orientation and spacing of three-dimensional elements.

FIG. 7 shows a sequence of steps wherein the method directly intakes or computes a rotation angle and axis for a three-dimensional element.

FIG. 8 shows steps that place strands broadly among centers, vertices and faces of elements as a whole.

FIG. 9 shows steps that pinpoint strands at centers, vertices and faces of three-dimensional elements.

FIG. 10 shows steps to determine length scale and shape among the strands.

FIG. 11 shows steps to intake and store functions and parameters that compute shapes of strands.

FIG. 12 shows steps that set up three dimensional elements and strands, and then compute paths and curvature of strands without interaction with strain.

FIG. 13 is an overview of steps that compute the number and spacing of loops in strands.

FIG. 14 shows an example of an η₁, η₂, η₃ coordinate system on a face of a three-dimensional element that rotates.

FIG. 15 shows a strand whose shape is affected by contact stresses from a three-dimensional element that rotates nearby.

FIG. 16 shows a strand whose shape at a particular point is determined both from a contact stress and from a frequency parameter of a previous sampling point.

FIG. 17 is an overview of steps that compute both twisting and non-twisting strain from an element that rotates.

FIG. 18 shows both interaction and separation of two strands based upon paths of the two strands.

FIG. 19 shows steps to compute, track and store interaction and separation data for two strands.

FIG. 20 is a schematic of emission and acceleration in a number of loops in a single strand as it interacts with contact stresses from rotation of different elements.

FIG. 21 shows steps that model emission and acceleration based upon curvature of a strand at a plurality of points.

FIG. 22 is a schematic of inflection points and extent of a strand in one coordinate direction.

FIG. 23 shows steps to compute a weighted density of inflection points to model a mass based upon density of inflection points.

FIG. 24 shows one strand that represents a wave that interacts with a path of a second strand.

FIG. 25 shows steps that track separation and interaction of a strand with a wave.

### DETAILED DESCRIPTION

In one mode of the invention, centers, vertices, edges and faces of three-dimensional elements are utilized to outline and enclose any one or more of atoms, atomic clusters, portions of lattices and material grains. Knots and strands are utilized to represent atoms, electrons, and nucleons. The shapes of the knots and strands change with rotational strain and interact with each other. Loops, inflection points, extents, and paths of strands then model both known and desired behavior in existing and new micro structures. One embodiment of this mode is a method to model any one or more organic molecules. A second embodiment of this mode is a method to model any one or more of atomic clusters, portions of lattices and material grains of an inorganic material.

In another mode of the invention, the three-dimensional elements and their strands are used as a mapping. Three dimensional elements, along with their orientation, rotation, and rotational strain map one set of data onto a set of information about the original set of data. The sensitivity of rotational strain, loops, spacing of loops, inflection points, extents and paths to original shape, orientation and rotational angle of the elements, along with sensitivity to intervals and increments in sampling of strands, creates a mapping of a set of data to derive a set of detailed information about said data. One embodiment of this mode is a mapping of any one or more of torque, speed, and energy consumption of a machine onto three dimensional elements and strands to yield a set of information that indicates a source of control or alteration of said machine. A second embodiment of this mode is a mapping of any one or more of nucleons, atoms, and electrons onto strands to yield information on the behavior of said nucleons, atoms, and electrons when their environment changes to cause rotation of said three dimensional elements. A third embodiment of this mode is a mapping of material grains or particles onto said three dimensional elements to yield a set of information about any one or more of rotational strain, stress, potential energy, and electronic structure when said three dimensional elements rotate.

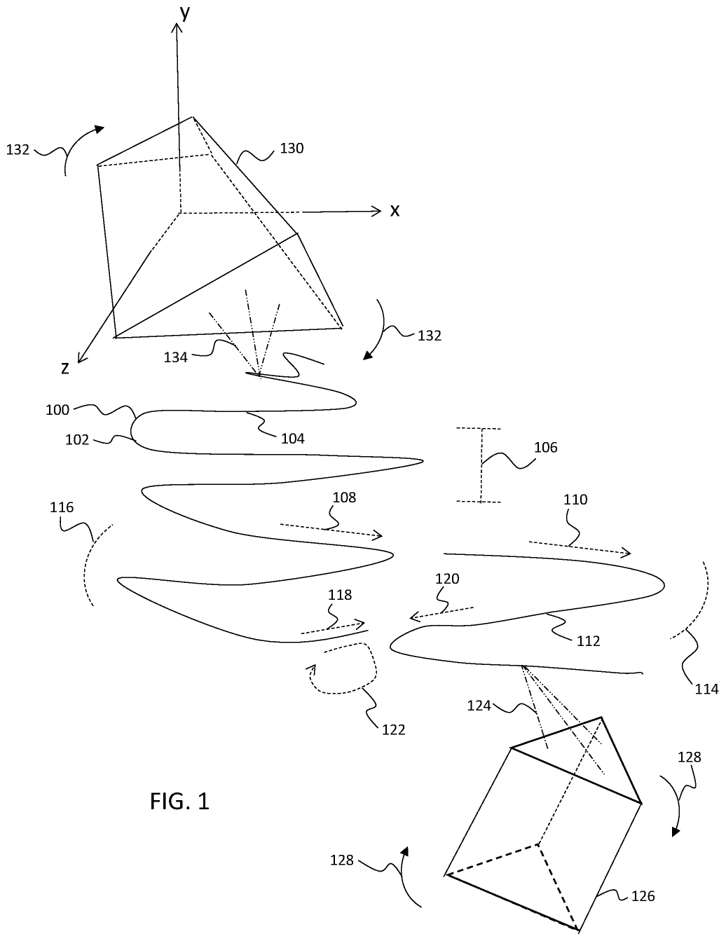

The present invention will now be described with reference to the accompanying drawings. FIG. 1 shows an overview of two strands that interact both with each other and with strain from rotation of two different three-dimensional elements. A strand 100 is represented by a succession of points in space whose coordinates in three orthogonal directions are computed by functions that contain cosines and sines. The functions can be summations of cosines and sines with differing amplitudes, frequencies, and phases. Alternatively, the functions can take on other forms that contain cosine and sine functions. Strand 100 can thus take the form of a knot, wave, or a combination of a knot and extended wave described by a single function.

Strand 100 in FIG. 1 can include one or more loops 102 and can include one or more inflection points 104. A loop is any curved portion between two inflection points. A loop can also be simply defined as a portion of a strand where its curvature increases or decreases in comparison with curvature at one or more previous point in space. The present invention can count a number of loops within a succession of sampling points and can compute a spacing 106 between loops. A spacing between loops can be a spacing between two loops or an average spacing of a plurality of loops. A loop 102 can be open, or it can be closed in a sense that a path of strand 100 crosses over itself.

In FIG. 1, a path 108 of a strand 100 is a vector whose direction and magnitude are determined by the present invention as a difference between coordinates of two successive sampling points in strand 100. Coordinates of sampling points in strand 100 are determined by functions dependent upon a parameter that is successively incremented. A path 108 of strand 100 can be in the same direction as a path 110 of a second strand 112. The two strands 100 and 112 can then move together in a loop with curvature 114 and curvature 116. A path 118 of strand 100 can also be in a direction that is opposite to the direction of a path 120 of strand 112.

In an embodiment in which strands represent successive amounts of potential energy in space, for example, a strand 100 might curve and move away in a path 122 from strand 112 so as to minimize an amount of energy contained in a small volume of space. The criteria for which two strands move in concert or separate from each other can vary, but the present invention can track paths of two strands and count the number of times paths could move in concert or separate. The result is a percentage of interaction or separation between two strands.

Strand 112 in FIG. 1 can have any one or more of its amplitude, frequency and phase parameters become dependent upon twisting and non-twisting strain terms that arise. A twisting and non-twisting strain in this description simply refers to those shear strains in the plane of a face that are torsional and non-torsional, respectively. A summation across a face also computes tensile and compressive stresses. Element 126 has planar faces that provide a contact stress within a medium within which it rotates. The summation 124 over a face provides strain at a particular point due to said contact stress. The amplitude, frequency and phase parameters that can become dependent upon said strain are within cosine and sine functions that partially or fully determine the shape of strand 112. Although element 126 shown in FIG. 1 has end faces outlined by triangles, element 126 can have any symmetry, size, or orientation. Similarly, rotation angle 128 can have any magnitude and can be about a rotation axis with any orientation and with any location within or on element 126.

Similarly, in the vicinity of strand 100 in FIG. 1, element 130 can rotate through an angle 132 to provide a contact stress with its planar faces. The present invention can perform a summation 134 over a face to compute twisting and non-twisting strain components. In a manner chosen by a user, the method can then link said strain components to amplitude, frequency and phase components in cosine and sine functions that partially or fully determine the shape of strand 100. As will be seen in subsequent sections of this description, the method can intake rotation angle 132 directly or it can compute the rotation angle from stresses applied to end faces of an element. A user can also decide not to have an element rotate at all. A user can then simply use an element to place one or more strands at desired locations.

One set of equations with which the present invention can compute coordinates of a succession of points that create a strand is

X=A₁ cos(ω₁t+p₁)+A₂ cos(ω₂t+p₂)+ . . . +A₃ sin(ω₃t +p₃)+A₄sin(ω₄ t+p₄)+ . . .

Y=B₁ cos(β₁t +q₁)+B₂ cos(β₂t +q₂)+ . . . +B₃sin(β₁t +q₁)+B₄ sin(β₄t +q₄)+ . . .

Z=C₁ cos(γ₁t+s₁)+C₂ cos(γ₂t+s₂)+ . . . +C₃sin(γ₃t +s₃)′C₄ sinsin(γ₄t+s₄)+ . . .

where X, Y and Z are orthogonal coordinates of successive points that are sampled in this invention to characterize a strand and its interaction with strain and with other strands. In the equations above, t is a parameter that is incremented over an interval that is usually the period of the cosine or sine function with the lowest frequency, but can be any other interval. In the equations above, the sets of terms Aᵢ, Bᵢ and Cᵢ, ωᵢ, βᵢ, and γᵢ, and pᵢ, qᵢ and sᵢ, where i is 1, 2, 3 . . . , are amplitudes, frequencies and phases, respectively, of the cosine and sine terms that may be present any number of times or not present at all in a computation of X, Y and Z at a particular point.

The present invention can use other sets of equations for three orthogonal coordinates that result in knots that can be highly asymmetrical and computed with a single set of equations. An example of such coordinates is

X=A(1+cos(ω₁t+p₁))cos(1/(ε+1+cos(ω₁t+p₁))t

Y=B(1+cos(ω₂t+p₂))cos(1/(ε+1+cos(ω₂t+p₂))t

Z=C(1+cos(ω₃t+p₃))cos(1/(ε+1+cos(ω₃t+p₃))t

In the equations above, A, B and C are amplitude terms, ω₁, ω₂ and ω₃ are frequencies, and p₁, p₂ and p₃ are phases. ε is nonzero adjustable parameter, and t is again a parameter incremented over an interval sufficient to characterize both the shape and interaction of one or more strands. In the equations above, the term 1+cos(ωᵢ+pᵢ), where i is 1, 2 and 3, modulates the amplitude of the cosine term that traces out the coordinates X, Y and Z.

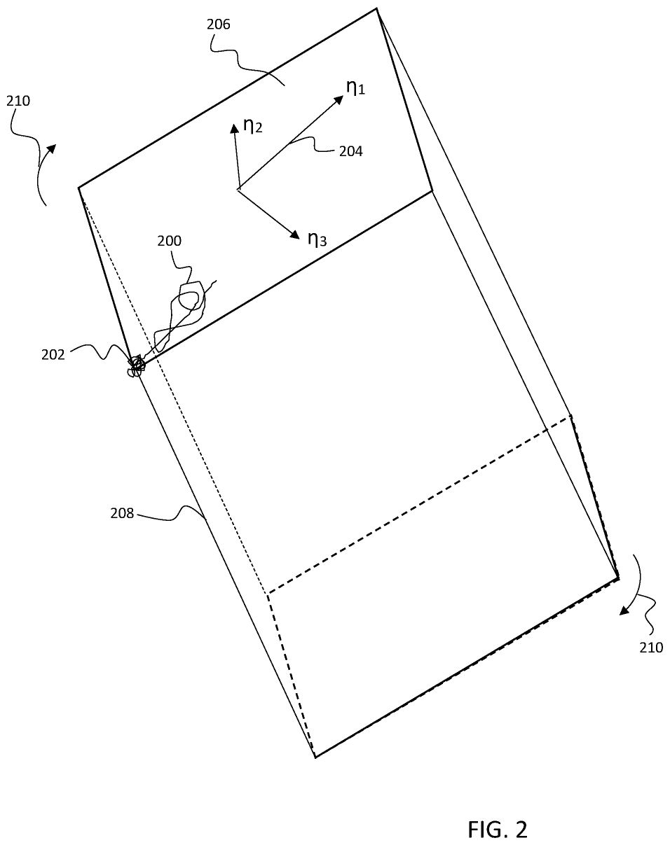

The term 1/(ε+1+cos(ωᵢt+pᵢ), where i is again 1, 2 and 3, gives a variable frequency to the cosine term. The result, shown schematically in FIG. 2, is a knot which has a slowly varying portion 200 with large amplitude that connects to a smaller portion 202 of more rapid frequency.

FIG. 2 shows a second orthogonal coordinate system in which the method can compute strain and then track strands for path, loops, and inflection points. In this η₁, η₂, η₃ coordinate system, the direction η₁, shown as element 204, is in the plane of a face 206. Direction η₁ is perpendicular to the rotation axis of an element 208 that rotates through an angle 210.

The method intakes data with regard to desired locations of strands in a model of a micro structure or in a mapping of data. Such intake is a succession of steps in which a user is prompted to indicate whether strands will be at one or more vertices, at one or more faces, or in the center of an element. The present invention is not limited to these prompts and intake with a user, but can function within the spirit and intent of the invention with another type of more rapid intake of data. The invention can also function with data that has been previously set before the start of a program.

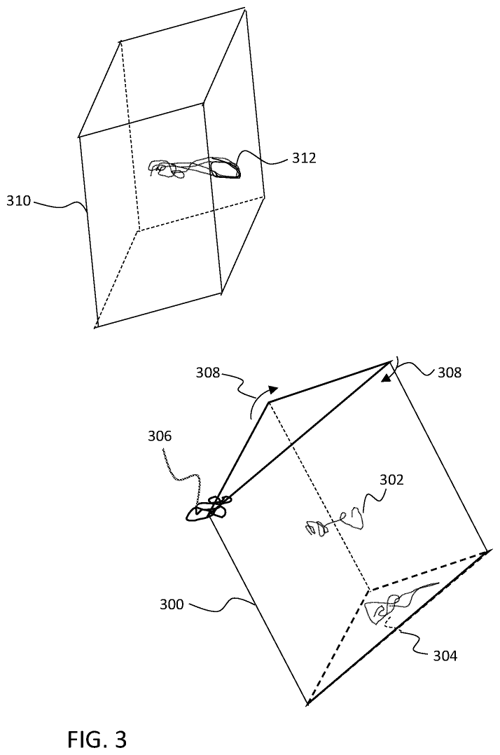

FIG. 3 shows examples of strand locations. For an element 300 shown in the lower portion of FIG. 3, a strand 302 may be in the center of element 300. A strand 304 may be at the center of mass of a face of element 300, and a strand 306 may by at a vertex of element 300. As will be shown in subsequent Figures and discussed in later sequences of steps in this description, an element 300 can have strands at none or at any number of its vertices and faces. Element 300 may or may not have a strand at its center. The various strands that might be present among the center, vertices and faces of element 300 may also have same or different shapes, and may have same or different length scales.

In FIG. 3, element 300 rotates within a shearable medium through an angle 308. Strands 302, 304 and 306 shown in FIG. 3 have shapes dependent upon initial parameters in the functions depicting their shape and upon strain from a contact stress that is a result of rotation of element 300 through said angle 308. Element 310 in FIG. 3, in contrast, does not rotate. Element 310 simply provides a location at its center for strand 312. Although element 310 does not rotate, strand 312 may have a shape that is influenced by strain from rotation of element 300.

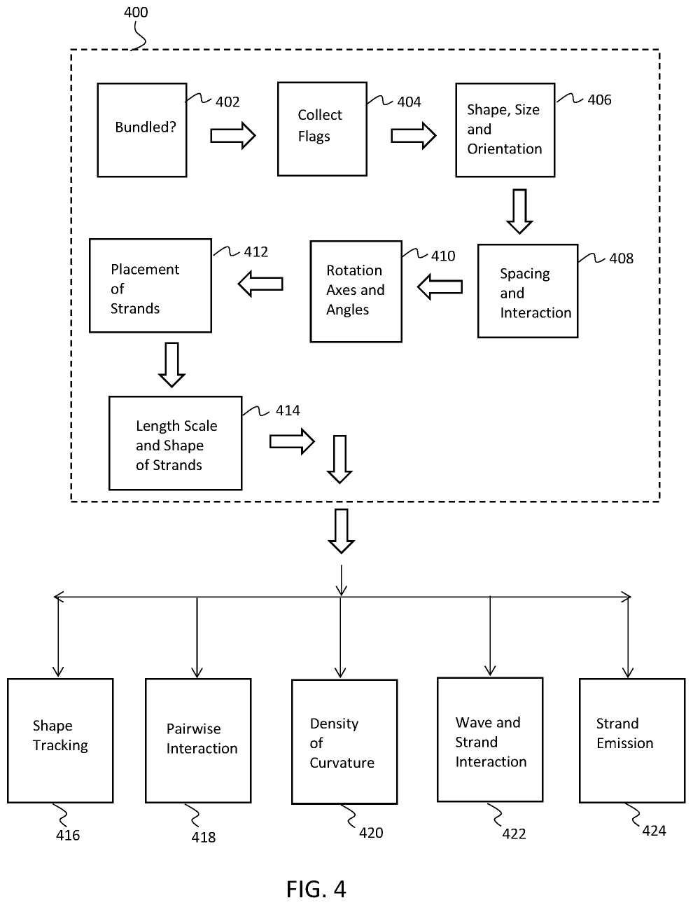

FIG. 4 is an overview of steps in the method that sets up a model of a micro structure and characterizes strands placed into said micro structure. The collection of steps 400, which are present in current programs within the method, set up the 3D elements and their strands. Different programs within the method, shown at the bottom of FIG. 4, will then move on to characterize strands and their interaction depending upon an intent and application of a particular program.

Within step 400 shown in FIG. 4, step 402 intakes data as to whether elements are bundled or not. Elements are bundled if two or more elements share a face with an adjacent element along a straight line connecting the centers of said two or more elements. Step 404 collects flags that denote general properties of three dimensional elements in a model. If the elements are unbundled, such properties include a total number of elements. If the elements are bundled, flags of 1 or 0 are collected as to whether or not all the bundles have the same number of elements, have elements of the same symmetry, and have elements of the same size and dimensions.

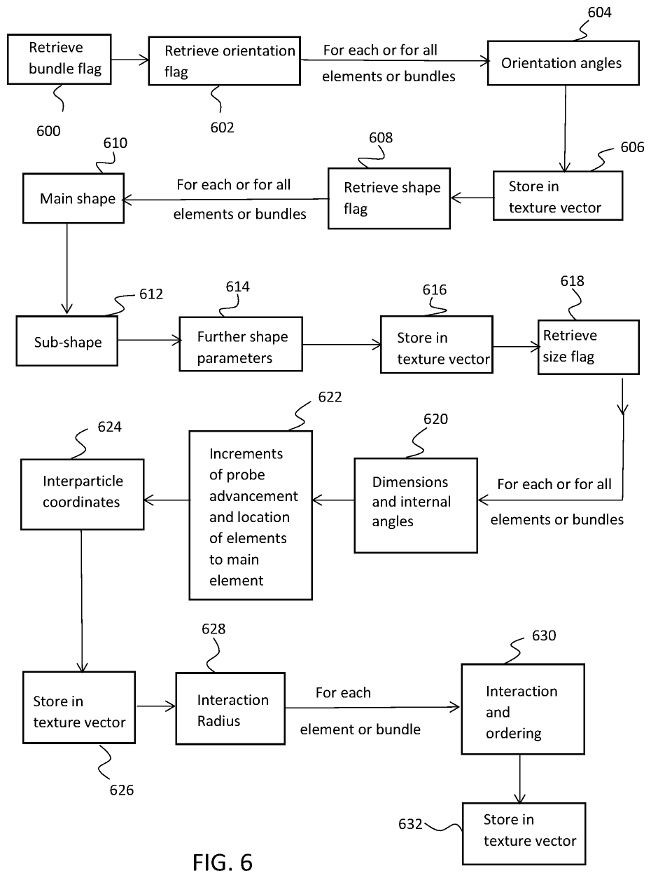

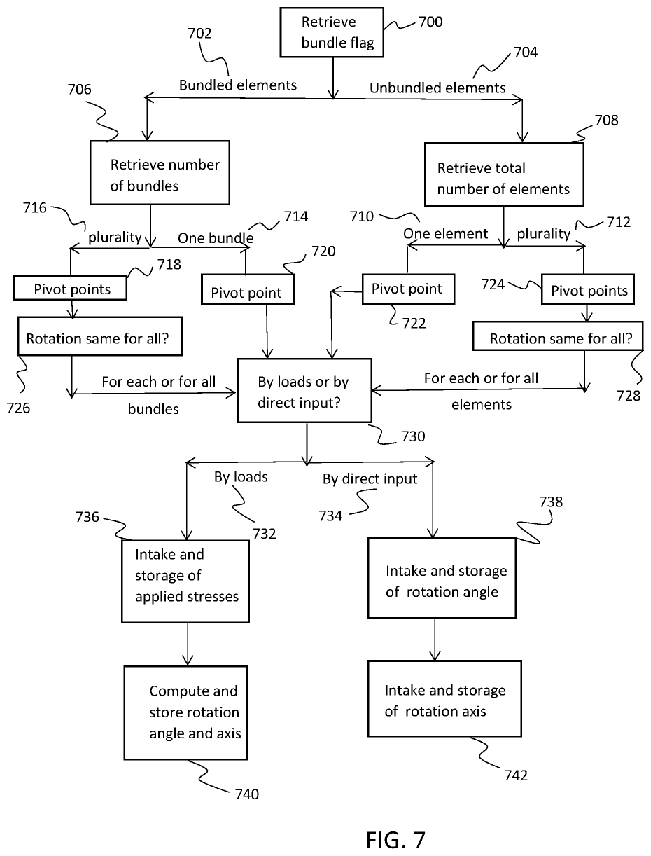

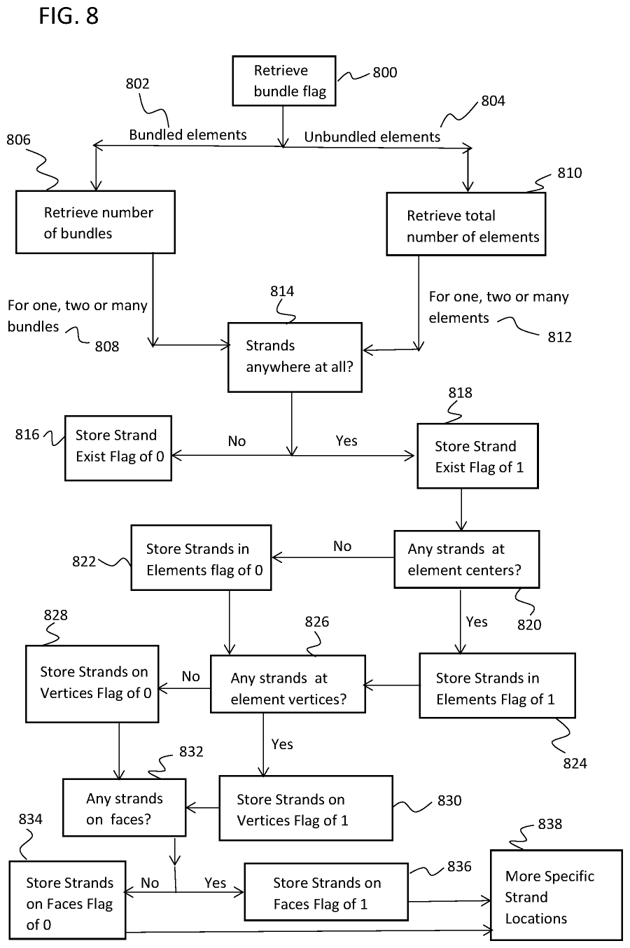

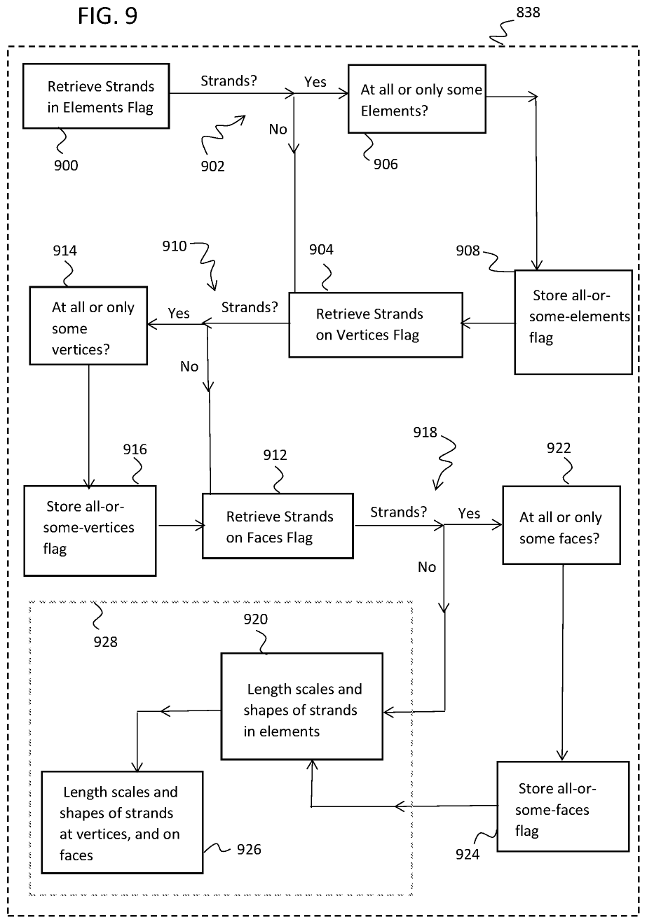

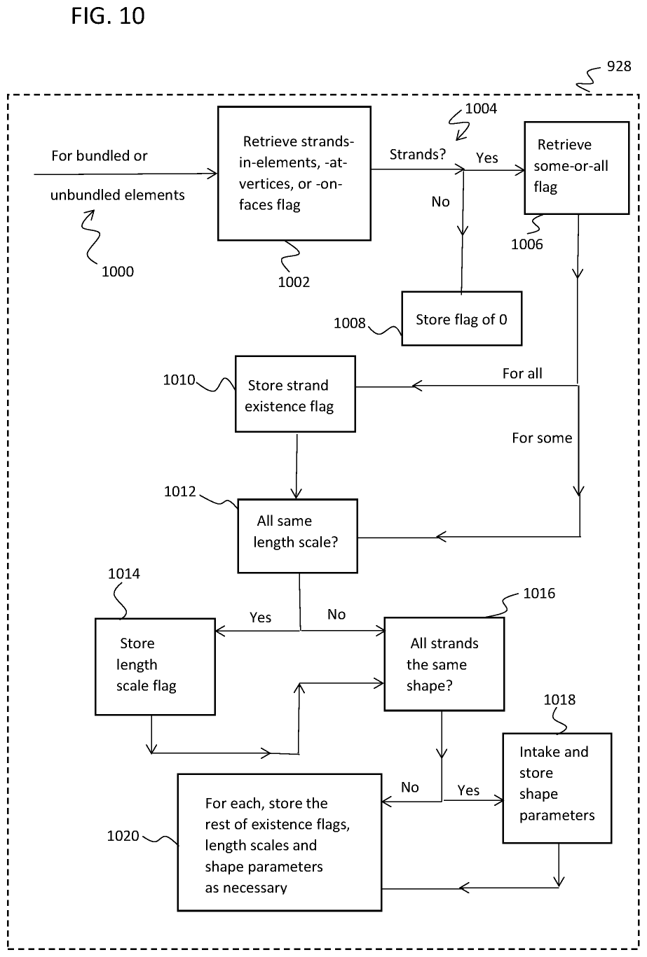

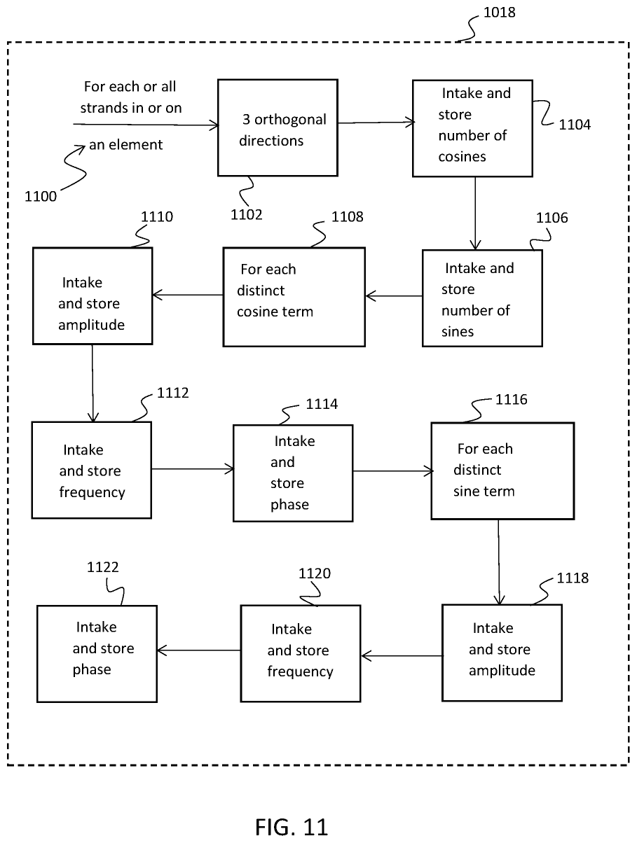

In FIG. 4, with use of the flags collected in step 404, steps 406 and 408 intake data with regard to symmetry, shape, size, orientation, spacing and interaction of the three-dimensional elements. Further sequences of steps for 406 and 408 are shown in FIG. 6 and are described in more detail later in this description. Step 410 in FIG. 4, which is described later in more detail with FIG. 7, intakes data for or computes both a rotation angle and direction cosines for a rotation axis for each three-dimensional element. Still one more step 412 in FIG. 4 intakes data with regard to placement of strands on the three-dimensional elements. Further detail for step 412 is shown in FIGS. 8 and 9. Step 414, which intakes length scales and parameters for shapes of strands, is described in more detail in step 928 in FIG. 9, and in FIGS. 10 and 11.

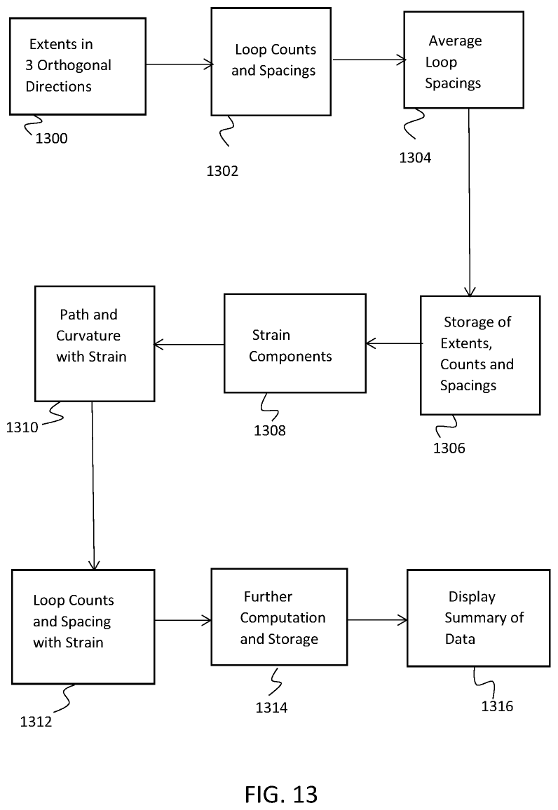

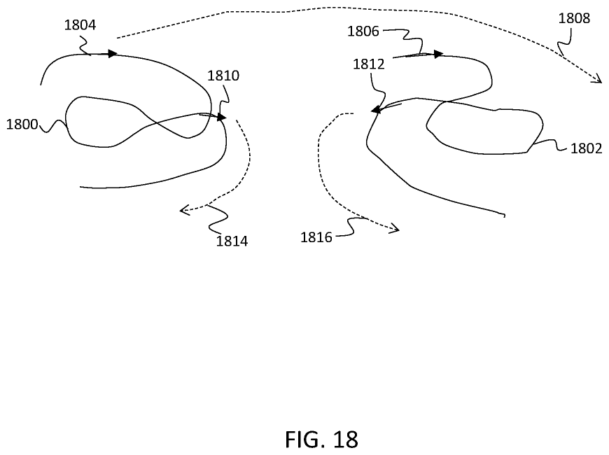

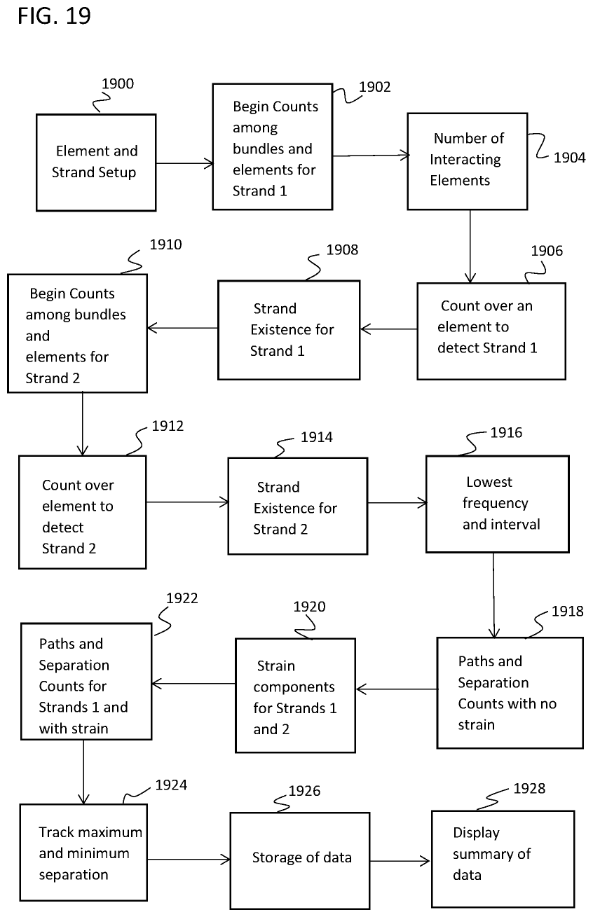

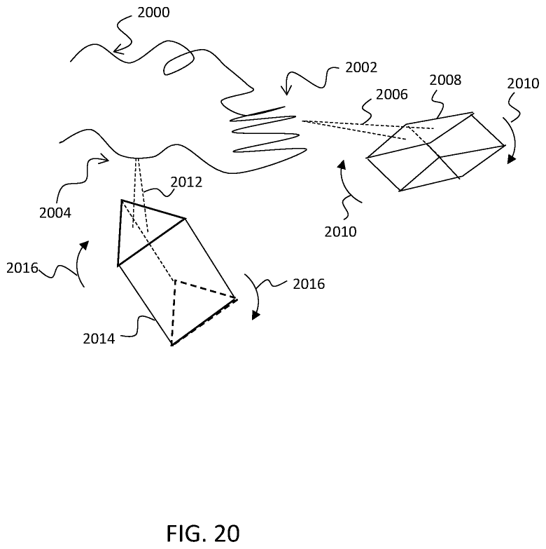

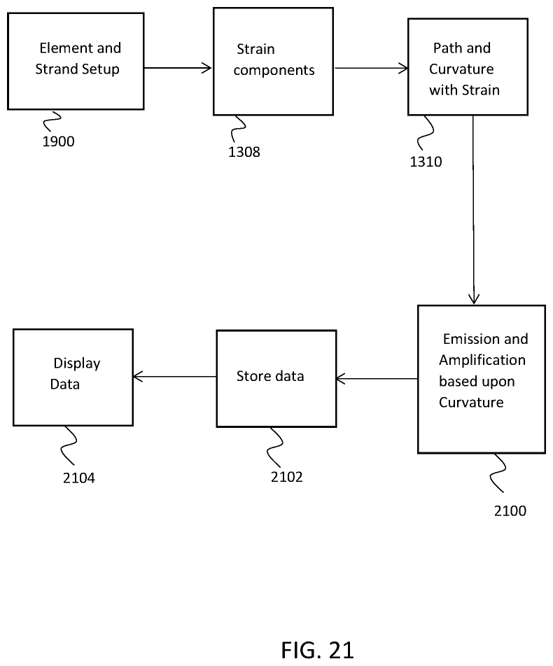

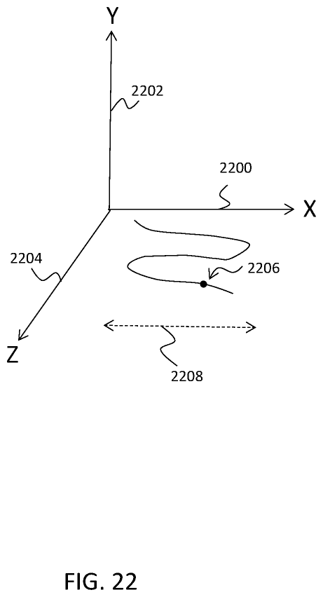

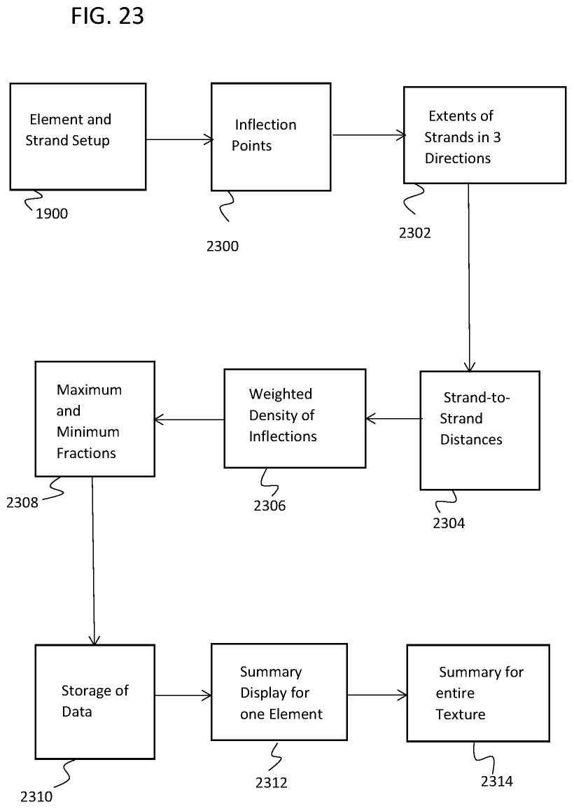

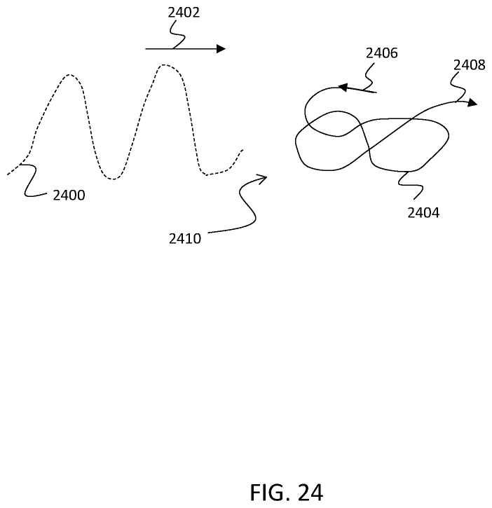

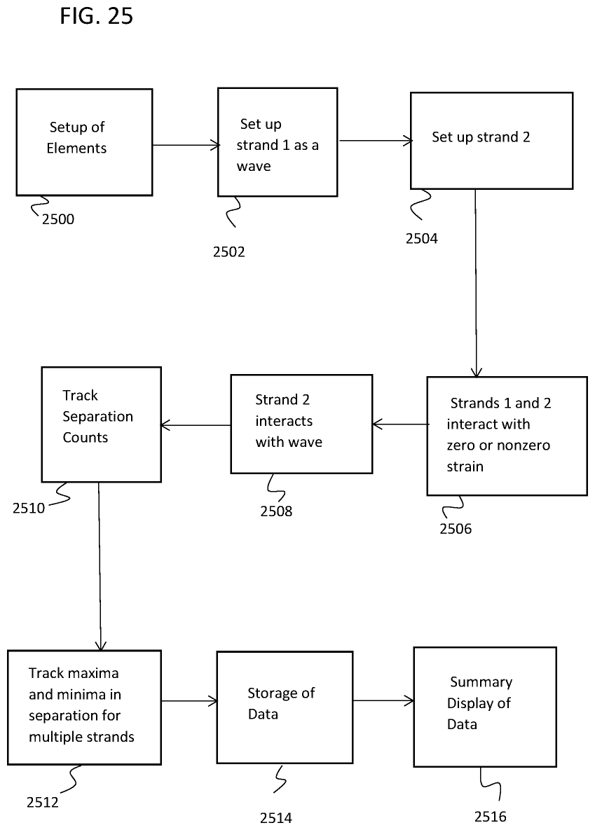

In FIG. 4, once step 400 is completed, the method includes several programs and any combination of these several programs that comprise further steps for various end uses. One program 416, for example, whose steps are shown in FIG. 13, tracks changes in curvature in strands to compute an average number of loops and spacing between loops in each strand. The same program 416 also tracks locations of sampling points to determine minima and maxima in extents of strands in three orthogonal directions. A program 418, which has more detail later in FIGS. 18 and 19, tracks paths of strands and supports pairwise interaction between strands. Another program 420, which also supports pairwise interaction between each pair of strands, tracks densities of loops in each of three orthogonal directions to model a shifting center of mass between each strand pair. More detail for program 420 is given later in FIGS. 22 and 23. Program 422, whose detail is described later with FIGS. 24 and 25, allows one strand simply to be a wave that interacts with other strands in a model. In still one more example, program 424, whose detail is in FIGS. 20 and 21, tracks values of curvature themselves to determine if a number of loops per path is increasing or decreasing.

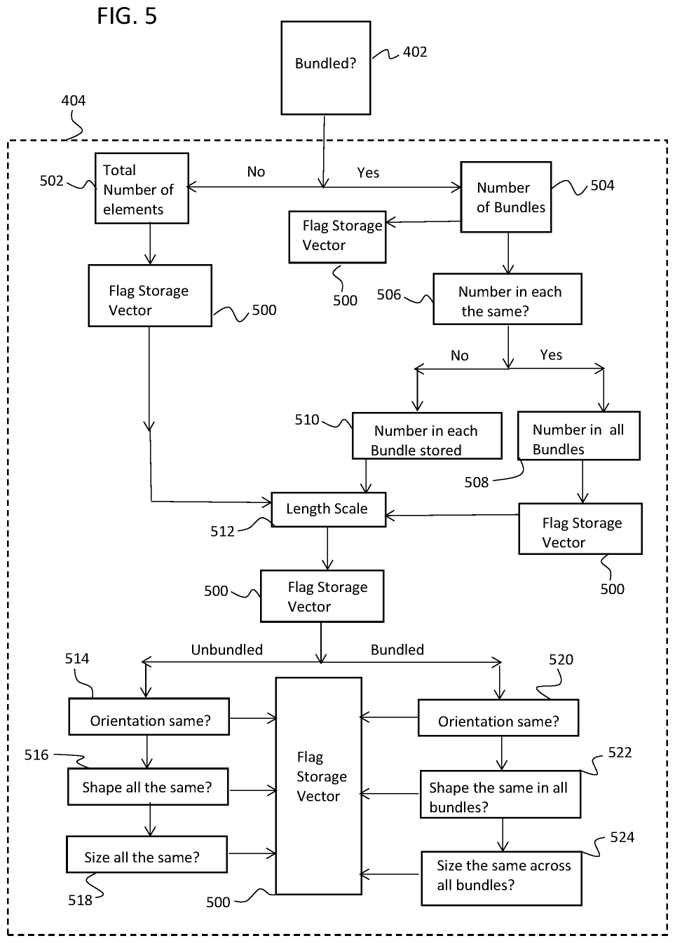

FIG. 5 shows more detail of step 404, which is intake of data and assignment of flags for the three-dimensional elements as a whole. The steps in FIG. 5 fill a two-dimensional vector container 500 called texture flags because flags and data in 500 describe a collection of three-dimensional elements, or texture as a whole. The container is not limited to a two-dimensional vector but can be another type of container or storage method.

Step 402 from FIG. 4, which is repeated at the top of FIG. 5 for reference, is intake as to whether the three-dimensional elements will be bundled or not. If the elements are not bundled, step 502 intakes from a user the total number of three-dimensional elements that will be used to set up a micro structure. The total number is stored in said two-dimensional vector 500. If the elements are bundled, the method intakes a number of bundles 504 and stores said number in said storage container 500. The method then intakes another flag 506 of value 0 or 1 as to whether each bundle has the same number of elements. If the number of elements is the same in each bundle, the method intakes the common number of elements 508 and stores said common number in said storage container 500. If the number of elements is not the same in each bundle, step 510 intakes the number of elements in each bundle and stores this number for each bundle in an array that is a storage container that is different from 500.

In FIG. 5, whether or not elements are bundled, the method performs a step 512 that intakes a length scale for a micro structural model as a whole. Step 512 intakes a flag of 1, 2 or 3 as whether the length scale is microns, nanometers or angstroms respectively, and then stores the flag in storage container 500. The method then branches again according to whether elements are bundled or not. If elements are unbundled, the method intakes a flag 514 that is 1 or 0 as to whether all the elements have the same orientation. Orientation, which is discussed again with FIG. 6, is a final result of rotating an element about the z, x, and y axes in succession. For unbundled elements, the method will also intake a flag 516 that denotes whether all the elements have the same shape. In the next step 518, intake of a flag of 1 or 0 indicates whether or not the elements are all the same size. The orientation, shape and size flags collected in steps 514, 516 and 518 are then stored in said storage container 500.

If elements are bundled, elements belonging to a particular bundle have the same orientation, size, and shape. Step 520, however, collects a flag of 1 or 0 as to whether all the bundles have the same orientation. In a similar manner, step 522 collects a flag of 1 or 0 as to whether all the elements have the same shape from one bundle to the next. Finally, step 524 collects a flag that denotes whether elements all have the same size from one bundle to the next. Flags collected in steps 520, 522 and 524 are also stored in said storage container 500.

FIG. 6 shows a sequence of steps used to set up the texture of the three-dimensional elements. By texture it is meant that the orientation, shape and spacing of the three-dimensional elements is set. The texture might be a pattern of atomic clusters or material grains, or it can be a set of three-dimensional elements, some of which overlap with one another. This latter pattern of three-dimensional elements results when some elements are used strategically to locate knots and strands in desired places.

In FIG. 6, step 600 first retrieves from storage container 500 the bundle flag that was set in step 402. The method then moves into a particular sequence of steps based upon whether elements are bundled or not. For either program branch, step 602 retrieves from storage container 500 the orientation flag that was set in either step 514 or 520. The orientation flag then creates two more program branches that in step 604 intake orientation angles for each element or bundle separately or for all the elements or bundles at once. In step 604, a first orientation angle is collected by rotating an element or bundle counterclockwise about the z axis, which extends straight out toward a user. A second orientation angle is collected in step 604 by rotating an element or bundle toward a user, or counterclockwise about an x axis, which extends to the right. Finally, a third orientation angle is collected in step 604 by twisting the already oriented element or bundle counterclockwise about a y axis, which extends upward. The present invention is not limited to this particular sequence of rotations, or to rotations about the x, y, and z axes to orient the elements. In the spirit and intent of the invention, other axes or rotation sequences may be used to orient the planar faces of the three-dimensional elements. An intent of said orientation angles is to provide distinct contact and rotational stresses from the three-dimensional elements. In one mode, the distinct rotational strains and stresses can provide a distinct mapping from one set of data to another set of information. In another mode, the rotational stresses interact with strands and knots that can represent micro structural elements or contours of potential energy.

In step 606 in FIG. 6, orientation angles are stored in a vector storage container for each three-dimensional element. The vector storage containers for each element are then part of a larger four-dimensional vector that stores data for the entire texture or pattern of three-dimensional elements. The storage containers are not limited to a vector. Storage can be done with another method or structure that allows storage and ready access to characteristics such as shape, dimensions, orientation angles, rotation angle, and direction cosines of a rotation axis for each element.

Similar to step 602, step 608 in FIG. 6 retrieves a shape flag that denotes whether all elements have the same shape. The method then goes into the appropriate loop for each element, for all elements, or for all bundles and collects a flag of 0, 1, 2, or 3 . . . in step 610 that denotes a main shape or symmetry for an appropriate number of elements. Symmetry includes a kind of rotational symmetry an element might have, or whether there is simply one mirror plane. In step 610, a main flag for a main shape is collected with respect to the crystal system the main symmetry elements belong to, such as monoclinic, tetragonal, cubic, etc.

In FIG. 6, sub-flags are then collected in step 612 to denote further shape delineation. Examples are sub-flags 1, 2 or 3 that indicate whether an element is a tetrahedron, octahedron or cube within a cubic system. In step 612, as appropriate, other sub-flags in other systems indicate whether the end faces of an element are outlined by a parallelogram, an equilateral triangle, isosceles triangle, right triangle, or an obtuse triangle. A three-dimensional element may also be triclinic with end faces outlined by triangles. Still more sub-flags of 1 or 0 are collected in step 614 that indicate whether an element is tapered, and whether there are pyramidal endcaps on its end faces. Similar to step 606, shape flags from steps 610, 612 and 614 are stored in a storage container in step 616 for each element.

Similar to steps 602 and 608 in FIG. 6, step 618 retrieves the previously stored size flag that indicates whether all unbundled elements are the same size, or, if elements are bundled, whether all elements are the same size from one bundle to the next. The method then branches into a loop for each element separately, or for all elements or bundles at once. In step 620, the method collects dimensions and internal angles as appropriate for the shape of each element. Dimensions can include a width, depth, and length, or it can include, as appropriate, lengths of sides of triangles outlining end faces of an element before collecting an overall length of an element. In a case of a tapered element, a user can indicate a size of the area of the smaller lower face as a percent of the area of the larger upper face. Dimensions of the lower face are then calculated and stored for a user. Similarly, in a case of a pyramidal endcaps on an element, a user may indicate a height of the endcaps as a percent of the length of the main portion of the element without endcaps. The method then computes and stores the height of the pyramidal endcaps. Internal angles are collected as appropriate for right rhombic prisms and for internal angles of triangles that may outline end faces of elements.

Once shape and orientation data are collected, the method computes and stores coordinates of the center of mass of each face of each element. It also computes and stores direction cosines of a vector normal to and extending outward from each face. The method also stores other data for each element, such as the number of and which faces surround each vertex, and the number of and which vertices surround each face. In this last step, faces and vertices are simply numbered and the method stores by number which faces surround each vertex and which vertices surround each face.

In FIG. 6, steps to store data for each element in the texture vector occur frequently. Different elements can have different numbers of vertices and faces, which results in differing numbers of vectors full of information for each element. Hence another vector storage container, nearly as large as the texture vector, is also filled and updated. Like the texture vector, this second storage container is a four-dimensional vector that supports a two-dimensional vector for each element. Final vectors in each two-dimensional vector store integer indices that indicate where particular information begin and end for a particular element in the large texture vector. One vector, for example, might store indices where coordinates and other information for each vertex begin and end. Another vector might store indices where information for faces begin and end. Still other stored indices indicate where in the texture vector information regarding an element's rotation axis begin and end. This invention is not limited to two large vector storage containers to store and retrieve information, but can utilize in its intent other storage methods.

In FIG. 6, step 622 begins a sequence of steps that support interaction of elements with one another. In step 622, the method first intakes which element will be a main element. Locations of other elements are obtained by collecting coordinates of other elements with respect to the main element. In the case of bundled elements, the main element does not need to be in the center of a particular bundle. In step 622, in the case of bundled elements, the method first intakes which bundle will be the main bundle. There is then further intake from a user to assign a main element within this main bundle. Locations of other bundles are then obtained by collecting coordinates of the center of other bundles with respect to the main bundle.

Step 622 then moves on to intake coordinates of the main element with respect to a probe point. The probe point is controlled by an outer loop that encompasses other loops in a program. The method intakes the number of times said outer loop is repeated. During each repetition of the outer loop, the probe point make advance in space, or the probe point may remain stationary. If the probe point advances, its end coordinates after all repetitions of the outer loop are obtained from a user. The method then calculates, as appropriate, incremental changes in position of the probe point in the x, y, and z directions along a straight line between its starting and ending points.

Different preferred modes of this invention have different uses for the probe point. In one mode, as the probe moves in a straight line, a spatial region in a shape of a sphere centered on the probe point advances. The spherical region acts as a region of interaction in that strands located within the sphere can have their paths compared with one another. Strands located outside the sphere would not have interaction data calculated. In another mode, the probe point is a model for a tip of a crack. As the tip of the crack moves, stress concentrations in front of the crack tip rotate one or more three dimensional elements. The strain from this rotation can then interact with strands in the vicinity. In yet one more mode, the probe point does not move at all. The outer counting loop that controls the probe point can still increment, however. For each outer loop increment, changes in position and density of loops in strands can model a strain that places new rotational forces on an element. When the outer loop increments, the result is a new rotation axis and angle of rotation for the element.

Step 622 also intakes coordinates of other elements and bundles, if any, with respect to the main element or to the center of the main bundle. Coordinates of each element or bundle with respect to the probe point are then stored in the texture storage container. In step 624, x, y, and z coordinates of all other elements with respect to each element are then computed. These interparticle coordinates are then stored in the texture storage vector in step 626.

Step 628 in FIG. 6 receives as input an interaction radius for each element. Within the interaction radius are the centers of other elements with which an element will interact. For bundled elements, a bundle will interact with other bundles whose centers are located within the interaction radius. For programs that support pairwise interaction between strands, strands on one element will interact pairwise with each strand on elements or bundles within the interaction radius. In step 630, the method counts through every element in the model and determines which elements that are within the interaction radius. Those that are within the interaction radius are then ordered by distance from the element at the center of a sphere with the interaction radius. In step 632, the bundle and element indices of each element within the interacting radius are then stored in the texture vector in order of distance from the central element. The storage of these indices facilitates retrieval of element information later if interacting elements are of interest in a program.

FIG. 7 shows steps the method takes to acquire and store information about the rotation of each element or bundle. In step 700, the method retrieves from the flags storage container 500 the flag that indicates whether elements in a model are bundled or not. The method then branches accordingly into routines 702 for bundled elements or routines 704 for unbundled elements. For bundled elements, in step 706, the method retrieves from storage container 500 the number of bundles in the model. For unbundled elements, the total number of elements would be retrieved from the flags storage container 500 in step 708.

In FIG. 7, if there is only one unbundled element present, the method will branch into routines 710 for one element. The method then continues in step 722 to intake a flag indicating whether the pivot point for rotation will be in the center of the element, or elsewhere on or in the element. The pivot point of an element is a set of coordinates in an orthogonal coordinate system indicating an intersection among a rotation axis, a plane of rotation, and the element itself. The pivot point can be in the interior of an element, at a vertex, along an edge, or on a face of the element. If it is not in the center, the method then intakes x, y, and z coordinates of the pivot point with respect to the center of the element.

In FIG. 7, if there is a plurality of unbundled elements, the method takes branch 712. Step 724 in branch 712 then includes intake of an additional flag that indicates whether all elements have their pivot point in the same location. If it is the same location for all elements, step 724 includes routines similar to those in step 722 that intake whether the pivot point is in the center of each element. The method then stores a location of 0, 0, 0, or else intakes and stores x, y, z coordinates with respect to the center of the element for the pivot point for each element. If the pivot point is in a different location for each element, the method will cycle through each element to intake a position of a pivot point for each element.

If the elements are bundled, FIG. 7 shows that the method moves into branch 714 for one bundle or into branch 716 for a plurality of bundles. Intake and storage of a pivot point for each bundle then occurs in step 718 for a plurality of bundles, or in step 720 for a single bundle. Routines in step 720 for a single bundle are similar to routines described earlier for step 722 for an unbundled element, except that coordinates of the pivot point are with respect to the center of the one bundle, rather than with respect to any one element. Similarly, routines in step 718 for a plurality of bundles are similar to routines described earlier in step 724 for a plurality of unbundled elements, except that pivot points are with respect to the center of each bundle, rather than with respect to the center of any one element.

When there is a plurality of bundles or unbundled elements, FIG. 7 shows that after pivot points are assigned, the method intakes a flag in either step 726 for bundled elements, or step 728 for unbundled elements, that denotes whether rotation will be the same for all bundles or elements. Same rotation means that both the angle of rotation and all the direction cosines of the rotation axis with respect to three orthogonal axes will be the same for all bundles or elements. The rest of the steps in FIG. 7 will then be performed accordingly once for all bundles or elements, or in a recurring loop for each bundle or element.

Whether there is just one or a plurality of bundles or unbundled elements, FIG. 7 shows that in step 730 the method intakes yet another flag that denotes whether rotation of a bundle or element will be determined by loads or by direct input. Determining rotation means intaking or assigning, and then storing, an angle of rotation in radians and direction cosines of a rotation axis with respect to three orthogonal axes. If rotation will be determined by direct input, the method moves into branch 734 in which there is direct intake from a user of a rotation angle in step 738 and direct intake from a user of direction cosines of a rotation axis in step 742.

If the method is to compute rotation from applied load in branch 732 in FIG. 7, there is first intake of applied stresses in step 736. Intake of applied stresses means that loads in each of three orthogonal directions that act upon particular planes are acquired either from a user or by computation. In one embodiment, a change in number and position of loops of strands is used to model strain. From there, a stress is assigned from the strain which is used to calculate rotation of an element. Once stresses are acquired, they act upon faces, usually end faces of an element. In step 740, the method then computes and stores a rotation angle and direction cosines of a rotation axis based upon said stresses acting across particular oriented faces of an element.

At any number of element centers, vertices and faces, the method can place strands of same or different shapes, and can place strands of same or different length scales. FIG. 8 shows first steps the method takes to place strands in a model. In step 800, the bundle flag that the method acquired in step 402 is again retrieved from the flags storage vector. Depending upon whether the bundle flag is 1 or 0, the method then moves into branch 802 for bundled elements or branch 804 for unbundled elements. If the elements are bundled, the number of bundles that was acquired and stored in step 504 is retrieved in step 806. The method then branches according to whether there is one, two or many bundles in step 808 to facilitate a dialogue that intakes from a user the rest of the information in FIG. 8. Similarly, if the elements are unbundled, step 810 retrieves the total number of elements acquired in step 502. The method then branches in step 812 according to whether there is one, two or many elements.

Whether or not the elements are bundled, the method will intake a strand existence flag in step 814 shown in FIG. 8. The existence flag denotes whether a model of a structure will have any strands at all. Without strands, the method can still compute twisting and non-twisting strain around elements. In one embodiment, for example, the method can model 3D elements and stress near the tip of a crack as the elements rotate. In another embodiment, incoming forces can interact in a macroscopic sense with twisting and non-twisting strain as 3D elements rotate. If there are no strands, an existence flag of 0 is stored in step 816. If there are strands, the method moves into another branch and stores an existence flag of 1 in step 818.

FIG. 8 shows that if there are strands in the model, the method will intake in step 820 a flag that indicates whether a strand will be at any of the element centers. If there are no strands at any element centers, a flag of 0 is stored in step 822. If there is a strand at one, any or all element centers, a flag of 1 is stored in step 824. Whether or not any strands are at element centers, the method moves to step 826 to collect a flag that indicates whether any strands are located at any of the vertices of the 3D elements in the texture. If there are no strands on any vertices, a strands-on-vertices flag of 0 is stored in step 828. If one, two, or a plurality of vertices have strands, said strands-on-vertices flag is 1 and stored in step 830. Similarly, in step 832 the method will intake whether any faces at all have strands. A strands-on-faces flag will be stored as 0 in step 834 if there are no strands on faces, and as 1 in step 836 if there is a strand at the center of mass of one or more faces in the texture. The method will then move on to step 838, which assigns more specific locations for strands. Step 838 is diagrammed in more detail in FIG. 9.

Steps for placing strands in more specific locations in step 838 are shown in FIG. 9. Step 900 retrieves from the flags storage vector a strands-in-elements flag, whose intake was in step 820. At step 902, the method then branches according to whether any elements have a strand at its center. If there are no strands at any element centers, the method moves to step 904 to retrieve the strands-on-vertices flag whose intake was in step 826. If there are strands at element centers, FIG. 9 shows that the method intakes another flag in step 906 that indicates whether all elements or only some elements in a model will have a strand at its center. The flag is stored in the texture flags vector or other storage container in step 908.

FIG. 9 shows that the method moves on to steps to assign more specific locations at vertices and faces. The steps are similar to those taken for the element centers. In step 904, method retrieves the strands-on-vertices flag that was assigned in step 826.

In step 910, the method branches according to whether any strands are on any vertices. If there are none on vertices, the method will move to step 912 to retrieve the strands-on-faces flag whose intake was in step 832. If there are strands on any of the vertices, step 914 intakes whether all or some vertices have strands. The method then stores a flag with this information in step 916 before retrieving the strands-on-faces flag in step 912. The method branches in step 918 according to whether any element faces have strands. If there are faces with strands, there is intake of an all-or-some-faces flag in step 922 that indicates whether all or just some faces have strands located at the center-of-mass of each face. Storage of the all-or-some-faces flag in the texture flags vector or other storage container occurs in step 924.

FIG. 9 then shows step 920, which entails collecting length scales and shape parameters for any strands located in element centers. Similarly, step 926 intakes and stores length scales and shape parameters for any strands on vertices and faces of the 3D elements. Steps 920 and 926 together constitute step 928, which is shown in more detail in FIG. 10 for intake of length scales and shapes of all strands.

FIG. 10 shows steps that the method takes to intake a length scale and shape of each strand that exists in a model. Steps in FIG. 10 create a vector for each element center, vertex, and face in a texture. If there is no strand at a particular location, said vector simply contains a strand existence flag of 0. If there is a strand at a particular location, said vector stores a strand existence flag of 1 and then goes on to store a flag for a length scale of said strand, the number of cosine and sine terms for the function depicting the shape of the strand, and then parameters such as amplitude, frequency and phase for each cosine and sine term. One purpose of steps shown in FIG. 10 is to intake and store information rapidly when strands of a same length scale and shape are at all element centers in a model, for example. The steps in FIG. 10 intake length scale and shape parameters one by one for each strand when strands are only at select locations, or when strands have lengths scales or shapes that differ from one strand to the next.

In FIG. 10, Step 1000 again retrieves the bundle flag whose intake was in step 402. The method of steps then branches accordingly as to whether 3D elements in the model are bundled or not to retrieve flags in a correct location in a texture flags storage vector. The sequence of steps in FIG. 10 is essentially the same for strands located in element centers, at vertices, or on faces, except that the steps are in different counting loops for element centers, vertices, and faces. The next step 1002 is therefore shown once and retrieves in its respective loop the strands-in-elements flag, strands-at-vertices flag, or strands-on-faces flag whose intake was in steps 820, 826 and 832, respectively. The method then branches in step 1004 according to whether strands exist at any location in a set, with a set being elements, vertices or faces. If there are no strands at any location in a set, then a flag of 0 is stored for each location in step 1008. If there are strands, step 1006 retrieves the all-or-some-elements, vertices or faces flag whose intake was in steps 908, 914 and 922, respectively.

FIG. 10 shows a branch labelled for all if said all-or-some-elements, vertices, or faces flag from step 1006 is 1, or yes. If strands cover all element centers, vertices, or faces the method moves to step 1010 shown and stores a strand existence flag of 1 in a first position of a storage vector for each strand. The method then moves to step 1012 to determine whether all the strands are of the same length scale. Similar to the 3D elements, the length scale can be angstroms, nanometers, microns, or something larger for continuum modeling. If the length scale is the same for all the strands, step 1014 stores a numerical flag of 1 to 4 indicating the length scale in the storage vector for each strand. The method then moves on to step 1016 to intake whether all the strands are of the same shape. If all the strands have the same shape, the method moves to step 1018 to collect and store parameters in each of three orthogonal directions for the functions denoting the shape of the strands.

If not all elements, vertices, or faces have strands, the method moves into branch labelled for some after step 1006 in FIG. 10. The method will then still proceed with steps 1012 and 1016 to intake whether the strands that do exist all have the same length scale and shape, respectively. If either length scale or shape parameters among the strands are the same, such information has intake just once before proceeding with step 1020. In step 1020, the method then loops just enough times to intake whether a strand exists at a particular location, and to store an existence flag, a length scale, and shape information in a vector for each strand.

FIG. 11 shows step 1018 for intaking and storing shape parameters of strands in more detail. In the first step 1100 in FIG. 11, the method branches according to step 1016 that indicated whether shape parameters are the same for all strands at element centers, vertices or faces. If the shape parameters are not all the same, step 1100 begins loops that count through each element center, vertex, and face as appropriate to intake shape parameters. In the case of vertices and faces, there is intake of an additional flag that indicates whether all vertices and faces on a particular element all have the same shape. The method then intakes shape parameters all at once for strands on a particular element, or counts through each vertex or face on a single element, as appropriate.

Step 1102 in FIG. 11 then opens three loops sequentially to intake parameters for a function in each of three orthogonal directions that contributes to the shape of a strand. As discussed earlier in this description, such a function can be a simple summation of cosine and sine terms, or it can be another function that may contain a cosine or sine function. The three orthogonal directions can be global x, y and z directions, or they can be orthogonal directions in any other system. Step 1104 then intakes and stores the number of cosines that might be present in a function in a particular coordinate direction depicting the shape of a strand. Similarly, step 1106 intakes the number of sine functions that are present in the function. Based upon the number of cosines from step 1104, step 1108 begins a loop that counts among cosine terms. For each cosine term, the method intakes and stores an amplitude in step 1110, a frequency in step 1112, and a phase in step 1114. Similarly, in step 1116 the method opens a loop to count among sine terms, the number of which was stored in step 1106. For each distinct sine term that may be present in a function for strand shape, the method intakes and stores an amplitude in step 1118, a frequency in step 1120, and a phase in step 1122.

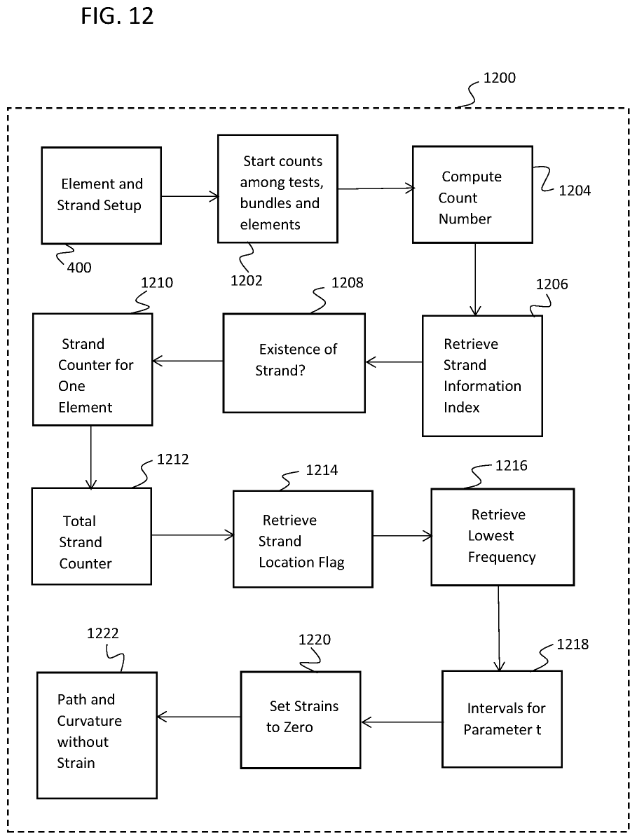

FIG. 12 shows a step 1200 that begins most embodiments of the invention. Step 1200 is the beginning, for example, of the programs labelled 416 to 424 at the bottom of FIG. 4. Step 1200 begins with step 400 that is a setup of 3D elements and strands. Step 400 is outlined in FIG. 4 and has the sub-steps shown in FIGS. 5 through 11. In step 1202, the method begins an outermost loop that counts a number of tests and for bundles and elements in a model. The loop for a number of tests can advance a probe point, a crack tip, or can begin a new rotation angle and axis, as in the earlier description for steps 736 and 740. The outermost loop is not limited to the examples given here but is simply an outer loop that can change the position or value of something and enclose other steps in a program. Step 1202 also begins counts among bundles and elements, which can be two sets of counts among two sets of bundles and elements when there is interaction between two strands in a program. In a case of pairwise interaction between strands, the method will open loops to count among those elements that are in a radius of interaction, as previously described for step 628.

Step 1204 in FIG. 12 then computes a count number based upon a sum of the number of vertices and faces an element has. The count number serves as an upper limit in a loop that counts through an element center and all vertices and faces of an element. Step 1206 then retrieves an integer index that indicates a location of the strand information storage containers that were filled in steps shown in FIG. 6 through 11. Step 1208 then checks a storage container for each element center, vertex, and face for the strand existence flag that was previously stored in steps 1010 or 1020. If a strand exists at a particular location, counters are incremented in step 1210 that count within a single element the number of strands, or the number of strand pairs, as applicable. If a strand exists at a particular location, other counters are incremented in step 1212 that count the total number of strands, the number of strand pairs, and the number of interacting strand pairs, as applicable, in an entire model.

For the strand that exists, step 1214 in FIG. 12 then retrieves a flag that indicates whether the strand is at the center, at a vertex, or on a face of the element at hand. From the information stored in step 1018, the method then retrieves in step 1216 the lowest frequency of the frequency parameters stored for the cosine and sine parameters of the function that traces out the strand. The lowest frequency is used in step 1218 so the intervals of a parameter t cover at least one period of the cosine or sine function with the lowest frequency in whatever direction that cosine or sine function might govern. The parameter t multiplies the frequency in the argument of each cosine and sine function. Although strains are later calculated from rotation of the 3D elements, they are initially set to zero in step 1220 so that characteristics of strands can be examined and recorded as they are traced out by their governing function alone without alteration from strain. A path of a strand is a direction determined by a difference in coordinates of two successive sampling points. In step 1222, the method computes path and curvature of a strand, counts inflection points in each of three orthogonal directions, and computes curvature of the strand as a whole.

FIG. 13 shows further steps, most of which are common to the programs labelled 416 through 424 at the bottom of FIG. 4. In step 1300, a function keeps track of minimum and maximum values of coordinates traced out for each strand in each of three orthogonal directions. A difference between final maximum and minimum values of a coordinate then becomes an extent of a strand in that direction. Using criteria in changes in curvature, a function in step 1302 counts loops. Step 1302 will also add up line segment lengths between each sampling point to approximate a total distance sampled. The total distance is then divided by the number of loops to get an average spacing between loops in step 1304. Storage of loop extents, counts and spacing occurs in step 1306 in such a manner that maxima and minima of loop extents, counts, and spacings for an entire collection of strands in a model are retrieved and displayed later.

In step 1308 shown in FIG. 13, strain components are calculated from rotation of a 3D element. In a current embodiment, the 3D element chosen to compute strain for each strand is nearest to a particular strand and has a rotation angle. The method would move on from an element such as 310 shown FIG. 3 that does not rotate. The method then finds an element that has a nonzero rotation angle that is nearest a strand at hand. Strain is computed from a contact stress made by a rotation of said nearest element within a shearable medium. Twisting and non-twisting strain components at a particular sampling point in said strand are computed from a summation over the element face that is nearest the strand. As components of strain from said rotation modify parameters in a function that traces out the strand, step 1310 calculates the path and curvature of the strand. Similar to the procedure previously described for steps 1302 and 1304, step 1312 in FIG. 13 counts loops of a strand and approximates loop spacing as the functions that are modified by said strains trace out the strand. Further computation and storage in step 1314 can include but is not limited to keeping track of maximum and minimum extents of the strand in each of three orthogonal directions, number of instances of very high or low curvature of a strand, or amount of separation or interaction between two strands depending upon the path of each strand. Examples of summaries of data displayed in step 1316 are amount of separation and interaction between each strand pair in a single element, and identification of strands with maximum and minimum extents in an entire model.

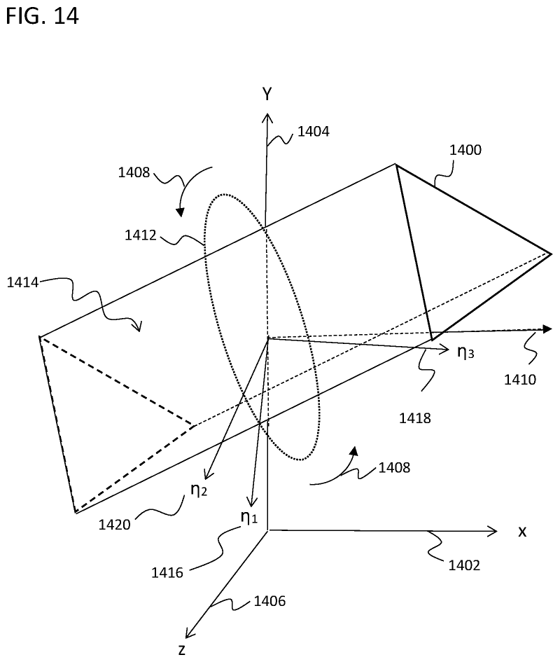

FIG. 14 shows a determination of the η₁, η₂, η₃ coordinate system that, as an example, is shown on the top face of the 3D element in FIG. 2. The η₁, η₂, η₃ coordinate system can be determined for any face on a 3D element and is an alternative to the global x, y, z coordinate system used to compute strain. Element 1400 shown as an example in FIG. 14 has triangular end faces, but in other embodiments can have any symmetry and orientation. The global x, y, z coordinate system for a model as a whole has an x axis 1402, a y axis 1404 and a z axis 1406 shown in FIG. 14. Element 1400 undergoes a rotation 1408 about a rotation axis 1410, which as an example is parallel to the x axis 1402 in FIG. 14. Rotation axis 1410 can have any orientation and is perpendicular to a rotation plane 1412 that is shown for clarity in FIG. 14 by a circle that lies in the plane of rotation 1412.

In the example in FIG. 14, the η₁, η₂, η₃ coordinate system is shown for face 1414, which is outlined by a rectangle, is in front, and nearly facing a viewer. The intersection between the plane of rotation 1412 and face 1414 then results in a line segment that is contained in the η₁ axis, which is labeled 1416. The origin of the η₁, η₂, η₃ coordinate system can be placed anywhere on the face, but in one embodiment is simply at the center of mass of the face. The η₃ axis 1418 is in the plane of the face and perpendicular to the η₁ axis 1416. The orientation of the η₃ axis can be set for convenience and does not necessarily make the η₁, η₂, η₃ system a right-handed coordinate system. The η₂axis 1420 extends from the origin and is perpendicular to face 1414 that contains the η₁ axis 1416.

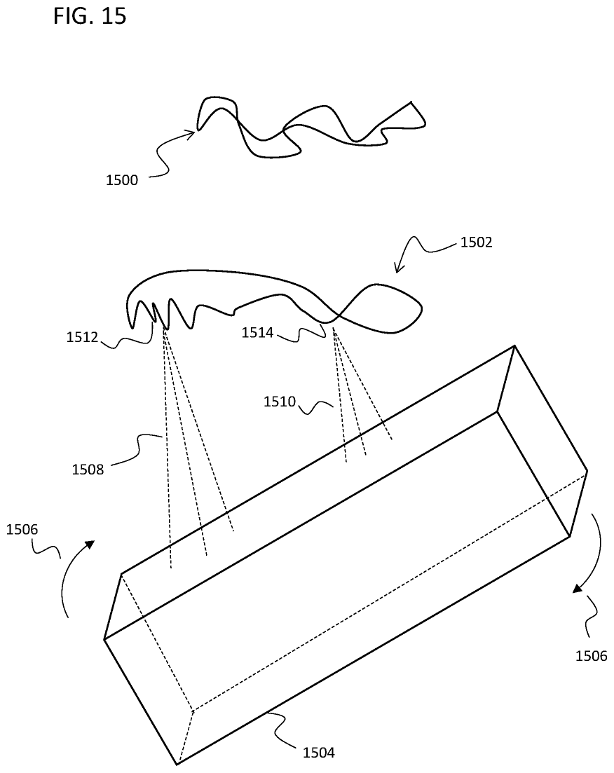

FIG. 15 shows one embodiment of a program labelled 416 in FIG. 4 in which the method characterizes a strand. The embodiment in FIG. 15 is useful when each point a strand is interacting only with the strain computed for that point. The method can count and track inflection points, loops, and extents of strand 1500, which is not interacting with any strain from a 3D element. Strand 1502, in contrast, interacts with strain that arises from an element 1504 that undergoes a rotation 1506. Rotation 1506 results in strain that differs from point to point on the face shown. Each value of strain on the face attenuates as it contributes to strain on a point located away from the face. Both the rotation of the element and attenuation of the strain result in strains 1508 and 1510, for example, that are computed by summing functions that represent strain across the face. A frequency 1512 in a function that traces out strand 1502 will be a result of the original frequency term in said function and a component of the strain 1508 that is affected by the distance of the strand from said face, the size of the face, the shape of the face, the orientation of the element 1504 and the magnitude and direction of the rotation 1506. Similarly, a frequency 1514 in a function that traces out strand 1502 at a different point in space will be a result of the original frequency parameter in said function and a component of strain 1510. Strain components 1510 differ from the set of strain components 1508 because 1510 has a different location with respect to the face along which summation occurs.

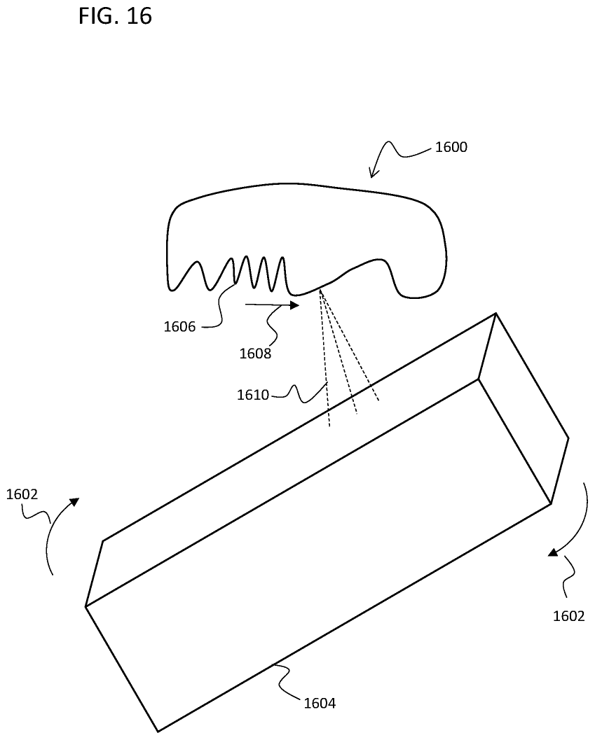

FIG. 16 shows an embodiment of program 416 in FIG. 4 in which the frequency parameter in a function that traces out a strand that varies with both strain and the frequency value at the last sampling point. The embodiment in FIG. 16 is useful when a strand might be tracked in time and for which parameters used to trace said strand vary with time. Strand 1600 is interacting with strain from a rotation 1602 of element 1604. A frequency parameter 1606 in a function that governs the shape of strand 1600 is then carried forward in step 1608 in the method to become the original frequency parameter at the next sampling point. At the next sampling point, frequency 1606 then interacts with a strain component that is a result of a summation 1610 of strains across the face nearest to said next sampling point. The new resulting frequency parameter will then in turn become the original frequency parameter at a next sampling point.

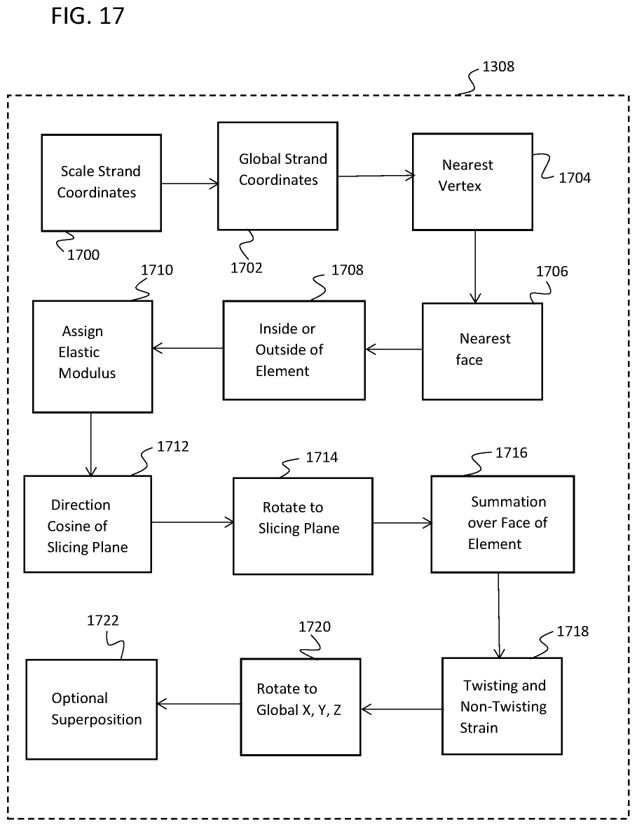

FIG. 17 shows detail of step 1308 in FIG. 13. FIG. 17 shows a sequence of steps in the method to compute strain from rotation of an element nearest a particular sampling point in a strand. The purpose of the first steps 1700 through 1706 in FIG. 17 is to determine the face nearest the sampling point at hand in a strand. The nearest face is used to sum over the face to add up the contributions to strain at the sampling point due to a contact stress that the face makes when its element rotates. When a function traces out a strand, it computes coordinates of a point in said strand about an origin for said strand in a length scale that may be the same or different from the length scale used for the dimensions of the elements around said strand. Hence the first step 1700 in FIG. 17, as necessary, converts the coordinates of the sampling point at hand to the length scale of the elements around it. The original or rescaled localized coordinates of the sampling point in the strand have an origin where the strand was first placed. A strand is first placed, for example, following steps in FIGS. 8 and 9, at an element center, vertex, or face. Step 1702 in FIG. 17 then converts these localized coordinates to the global x, y, z system whose origin is at the probe point. The probe point was described earlier for step 622 in FIG. 6. The global strand coordinates in step 1702 are then ready for use with global coordinates of the 3D elements to find a rotating element nearest the strand. A nearest element that rotates is found because the strain computed for interaction with the strand will arise from rotation of that 3D element. As described previously for element 310 in FIG. 3, a non-rotating 3D element can simply be used to place a strand at a particular point for convenience.

Step 1704 in FIG. 17 then retrieves coordinates for each vertex of the rotating element that is nearest said strand. The method then determines the vertex nearest said strand by computing and comparing distances from each vertex to the sampling point. The method then retrieves from the texture storage vector the number of faces that surround said nearest vertex and indices which indicate exactly which faces surround said nearest vertex. In step 1706, the method then retrieves from said storage vector coordinates of the center of mass of each face that surround said nearest vertex. By computing and comparing distance between each center of mass and the sampling point, the method determines which face is nearest the sampling point at hand in a strand.

Step 1708 in FIG. 17 moves on to determine whether the sampling point at hand in a strand is inside or outside its nearest rotating element. The method does this by transforming the global x, y, z coordinates of the sampling point at hand to a coordinate system that is local to the nearest face found in step 1706. In this localized coordinate system, the origin is at the center of mass of said nearest face, the y axis is perpendicular to the plane of the face, and the x and z directions are in the plane of the face. The x and z directions, which are chosen for convenience, are in most embodiments such that the x axis is parallel to one of the edges of the face. The method then assigns a flag indicating whether a sampling point is inside or outside an element based upon whether the y coordinate of the sampling point at hand is negative or positive in this localized system.

Step 1710 shown in FIG. 17 assigns elastic moduli, or proportionality constants, between stress and strain. Step 1710 is after step 1708 to support same or different materials inside and outside a particular 3D element. Step 1712 then determines the direction cosine between the “slicing plane”, or plane of rotation 1412 shown in FIG. 14, and the x direction in the localized face coordinate system described for step 1708. The direction cosine determined in step 1712 is also simply the direction cosine between the η₁ direction in FIG. 14 and the x direction in the localized face coordinate system described for step 1708.

For the sampling point, step 1714 then “rotates” its local x and z axes to the η₁ and η₃ axes to determine coordinates of the sampling point in the η₁, η₂, η₃ system. The method also computes and stores coordinates of vertices and parameters of equations of lines containing edges of the face in this same η₁, η₂, η₃ system. The method in step 1716 then performs a summation in the η₁, η₂, η₃ system over the face to account for each contribution of strain across the face for the sampling point at hand. During the routines for this summation, the method takes step 1718 to calculate twisting and non-twisting strain, which simply refers to strains resulting from circular and straight displacements respectively across a particular face as its element rotates.

In step 1720, strain components from step 1716 for the sampling point at hand are then rotated in most but not all programs back to the global x, y, z coordinate system. There is flexibility in the method in that some of its programs support characterization of strands with strain in the η₁, η₂, η₃ coordinate system, while other programs support characterization of strands with strain in the x, y, z coordinate system. In step 1722, some programs in the method superimpose a stress that is incoming and from the environment around an element with the stresses that are derived from said rotational strains from steps 1716 and 1718. Step 1722 is optional in that most programs simply modify shapes of strands directly with strain components from steps 1716 and 1718.

FIG. 18 shows pictorially the pairwise interaction of two strands that was mentioned in program 418 in FIG. 4. Two strands 1800 and 1802 can be either overlapping or separated in space. In FIG. 18, the path of a strand, which is represented by a vector drawn between two successive sampling points, has the same construction as the vector 108 that was defined in FIG. 1. In FIG. 18, path 1804 of strand 1800 can be in the same direction as path 1806 of strand 1802. When the direction of the two paths is the same, the method models any one or more of a path, arc, interaction and bonding 1808 of the two strands 1800 and 1802. Conversely, when a path 1810 of strand 1800 is in a direction opposite to path 1812 of strand 1802, strand 1800 can take a path 1814 and strand 1802 can take a path 1816. With paths 1814 and 1816, either separately or together, the method models a separation or repulsion between the two strands.

FIG. 19 shows a sequence of steps the method takes for an interaction between the two strands shown in FIG. 18. Step 1900 has a user set up three dimensional elements and strands in a micro structural model. Step 1900 has an overview 400 in FIG. 4, and includes steps described in FIGS. 5 through 11. FIG. 19 focuses on steps for a single strand pair, although there can be multiple strands and hence multiple strand pairs in a model that go through the steps in FIG. 19. Step 1902 begins loops that count through bundles and elements to find a first strand in a strand pair. For each element in step 1902, the method in step 1904 extracts from the texture storage vector the number of elements within its radius of interaction. The number of interacting elements around a particular element was described previously for steps 628 and 630 in FIG. 6. Use of data from step 1904 is optional. The method can find strand pairs either throughout a model or within a radius of interaction around each element.

Step 1906 in FIG. 1 retrieves the number of vertices and faces a particular element has. Step 1906 then counts over the vectors that store strand information for the element center, vertices, and faces. The filling of these vectors, with a strand existence flag in the first position, was described previously in the steps shown in FIGS. 8 through 10. Step 1908 retrieves the strand existence flag and checks if it is 1. If a strand exists at a particular position, the strand becomes strand number 1 in an interacting pair, and the method moves to step 1910. Similar to the steps just described to assign strand number 1, the method in step 1910 begins counting among bundles and elements for strand 2. In step 1912, the method again counts over the center, vertices and faces of an element but assigns and begins with a floor in the counting to prevent assigning strand 2 as the previously assigned strand 1. Step 1914 then retrieves and evaluates a strand existence flag stored for each potential location of a strand 2 and then assigns strand 2 when said existence flag is 1. The method is not limited to vectors as storage containers, nor is it limited with element centers, vertices and faces for the location of strands. Without departing from the scope and spirit of the invention, various changes and modifications can be made to store and to retrieve strand information, and to place strands at other locations among the three-dimensional elements.

Step 1916 in FIG. 19 then retrieves a lowest frequency for each of strands 1 and 2 from the frequency parameters in the cosine and sine functions contained within the functions that describe the shape of these strands. A lowest frequency and its use in assigning intervals and in incrementing a parameter tin strand functions was described previously in steps 1216 and 1218. The steps are done for both strands because an interval for t is assigned for each strand to ensure that sampling is performed over a portion of strand that will sufficiently characterize it.

In step 1918, with no strain components altering parameters in the functions that trace out the strands, the method tracks paths for both strands and increments a counter each time the components of paths of the two strands are in opposite directions. Paths were shown previously, for example, in elements 108 and 110 in FIG. 1. FIG. 18 shows an example of two paths 1810 and 1812 in opposite directions, with corresponding separations 1814 and 1816 in paths. Following step 1308, whose sub-steps were given in FIG. 17, step 1920 computes strain components for each of strands 1 and 2 by summing over a nearest face to each strand. Similar to step 1918, step 1922 then tracks paths and separation counts for each of strands 1 and 2 with parameters in the functions that trace out their paths modified by strain components found in step 1920.

Step 1924 keeps track of which strand pairs in a model have the most and least separation counts. The method computes a separation fraction by dividing the number of separation counts by the total number of sampling points. The method then stores in step 1926 a separation fraction in each of three orthogonal directions for each pair. Step 1926 also stores maximum and minimum separation fractions for all the strands in a model as a whole, along with the bundle, element, and location coordinates of the strands in the pairs with said maximum and minimum separation fractions. Step 1928 then displays a summary of data, which can include separation fractions for each strand pair within a particular element, and maximum and minimum separation fractions in the model as a whole.

FIG. 20 shows an embodiment of the method used for density of curvature in a strand in a program 420, or used for emission of a strand in program 424 in FIG. 4. A model for strand emission can take any of a plurality of forms and can include strand separation, as in FIG. 18, or variations in curvature, as in FIG. 20. In FIG. 20, strand 2000 has a region of high curvature 2002 that has a relatively large number of loops per unit volume. Strand 2000 also has a region of low curvature 2004 that has a lower number of loops per unit volume in comparison with region 2002. The function that traces strand 2000 in region 2002 has a frequency parameter that is affected by strain that is a result of summation 2006 over a face of element 2008 that undergoes a rotation 2010. Region 2004, with a smaller amount of curvature per unit volume, has a frequency parameter that is affected by strain that is a result of summation 2012 over a face of element 2014 that undergoes a rotation 2016. FIG. 20 is but one example of an embodiment of a strand whose curvature varies significantly from one region of the strand to the next. In other embodiments, elements 2008 and 2014 can have the same or different shapes. Elements 2008 and 2014 can also have the same or differing orientations in space and rotation angles. Strand 2000 with its variations in loop density can also interact with strains that result from summation over different faces of the same three-dimensional element.

FIG. 21 shows a sequence of steps the method takes to set up and analyze curvature in the strand 2000 in FIG. 20. The sequence begins with setup of three-dimensional elements and strands, as described for step 1900 in FIG. 19. The sequence can set up more than the one strand that is shown for simplicity in FIG. 20. From rotation of said three dimensional elements, the method then computes strain components, as described for step 1308 in FIG. 13, for each sampling point in a strand. In the next step in FIG. 21, the method modifies parameters in the functions that trace out the strands with the strain components from the preceding step. Path and curvature at each sampling point are then computed as described previously for step 1310 in FIG. 13. In step 2100, the method increments a counter for emission and amplification at each sampling point based upon curvature at that sampling point. If curvature is less than a set value, for example, the strand can be modeled as separating or emitting in space from a location it previously occupied. A counter for emission is then incremented by 1. If said curvature is greater than a set value, for example, a larger density of loops in the strand can be modeled as an amplification of a quantity that is represented by a density of loops in said strand. A count for amplifications is then incremented by 1. Fractions of a strand emitted and amplified can then be computed by dividing said counts by the total number of sampling points. The method then stores said fractions emitted and amplified in step 2102. It displays said fractions for the strand at hand in step 2104.

FIG. 22 shows a schematic diagram of a model the method creates for a center-of-mass based upon densities of inflection points in three orthogonal directions. FIG. 22 shows a tracking of inflection points in one direction for program 420 in FIG. 4. Although a few inflection points are shown for a tracing of a strand in the x direction, the method tracks inflection points as functions trace out a strand along the x axis 2200, y axis 2202, and z axis 2204. By tracking changes in curvature in the x, y and z directions, the method detects and counts an inflection point 2206 and any other inflection points in the x, y, and z directions during a sampling of a strand. The method also detects and tracks the maximum extent 2208 in the x direction for a strand. The method then models, as an example, a mass in each of the x, y, and z directions as a total number of inflection points in a particular direction divided by the total maximum extent of a strand in that same direction during sampling. Using strand-to-strand coordinates for a strand pair, the method can use a weighted mass fraction for the two strands to model and track a center-of-mass between two strands.

FIG. 23 shows a sequence steps the method can take to model a center of mass between a pair of strands. The steps in FIG. 23 track a density of curvature for program 420 in FIG. 4, and for the schematic diagram in FIG. 22. The first step 1900, an element and strand setup, has an overview 400 in FIG. 4, and includes steps described in FIGS. 5 through 11. In the next step 2300, the method counts the number of inflection points in each of three orthogonal directions as it samples points along a strand. A number of inflection points is related to a number of loops in a strand, and is hence related to a number of times curvature rises within an extent of space in a coordinate direction. In step 2302, as it samples a strand, the method keeps track of maximum and minimum coordinates in each direction. At the end of sampling, in each coordinate direction, a difference between maximum and minimum coordinates then becomes an extent of the strand in that direction. In step 2304, the method retrieves x, y, and z coordinates of two strands and computes strand-to-strand distances in each of the three coordinate directions. The strand-to-strand distances then serve as components of a vector between the two strands in a pair.

In step 2306, for each strand in a pair, the number of inflection points in each direction from step 2300 is then divided by the extent in each direction from step 2304 to compute a density of inflection points, or a model of a mass, in each direction. To model a mass fraction, the method takes the resulting inflection point density and divides by the sum of masses for both strands in that coordinate direction. The method in one embodiment can then model center-of-mass coordinates between the two strands by multiplying the weighted density of inflection points in a particular direction by the corresponding component of the vector between the two strands.

In step 2308, the method keeps track of maximum and minimum mass fractions to determine which strand pairs have a modeled center-of-mass shifted away the most between a midpoint between two strands. In step 2310, the method stores mass fractions, and center-of-mass coordinates for each strand pair, along with maximum and minimum mass fractions for the micro structure as a whole. In step 2312, for each strand in a single element, the method retrieves and displays data derived from pairs between that strand and any other strand in the texture. In step 2314, the method retrieves and displays maximum and minimum mass fractions for the micro structure as a whole, along with the elements and coordinates that belong to the strands in these pairs that have maximum and minimum mass fractions.

FIG. 24 shows an embodiment when one of the strands in a pair is a wave. Wave 2400 is traced by functions for x, y and z coordinates that describe a wave. One or more of the coordinates is set so that wave 2400 has a direction of propagation 2402 toward a strand 2404 that can be a knot, a strand like entity, or even another wave. For two successive pairs of sampling points, one pair for wave 2400 and one pair for strand 2404, path 2406 of strand 2404 can be in a direction opposite to the direction of propagation of wave 2402 as a whole. Path 2406 can also simply be in a direction opposite to a path in another coordinate direction described by wave 2400. The method can then increment a separation counter to model a wave and strand separating into opposite directions. Path 2408 of strand 2404 can be in the same direction of the direction of propagation 2402 of wave 2400. Path 2408 can also be in the same direction as the path of another coordinate described by wave 2400. In this case, wave 2400 and strand 2404 may move in concert in the same direction and a separation counter is not incremented. In the end, the method summarizes what fraction of the interaction between wave 2400 and strand 2404 is separation. Initially, or by interaction, region 2410 occupied by strand 2404 can be separate or overlapping with wave 2400.

FIG. 25 shows steps the method takes to model a wave interacting with the strand shown in FIG. 24. Step 2500 begins with a setup of one or more three dimensional elements, which was described earlier in the steps shown in FIGS. 5 through 7. Step 2502 utilizes existing functions or allows a user or the method itself to add new functions that allow a first strand 1 to propagate as a wave. Strand 2 is set up in step 2504 and includes steps described earlier in FIGS. 8 through 11. In step 2506, functions that trace out strand 2 can interact with zero or nonzero rotational strain, as desired. Step 2506, for example, can include step 1308 described earlier with FIG. 17, which calculates rotational strain from an element. Interacting with zero or nonzero strain means that strand 2 might alter its shape from strain or it might be traced out without strain. The wave, or strand 1, may be electromagnetic, a stress wave, a vibrational wave, or some other wave. The wave might interact with strain, or it might be traced out without strain affecting its parameters. Strand 2, which may or may not be a wave itself, then interacts with the wave in step 2508, which can include tracking the paths between sampling points to determine whether or not the paths of the strand and wave are in the same or opposite directions.

Step 2510 in FIG. 25 then tracks separation counts that can be modeled upon but are not limited to paths of the wave and strand. A fraction separated is a separation count divided by the number of points sampled for a particular strand pair. When there are multiple strands in a model, step 2512 goes further to track maximum and minimum fractions separated. In step 2514, the method stores which wave and strand pairs result in the most or least separation. Step 2514 also stores separation fractions between a wave and each of a plurality of strands within one element. The final step 2516 displays separation fractions for each wave-strand pair within each element. Step 2516 goes further to display the indices of the bundle and element that contains the strand, the location of the strand with regard to the center, vertex, or face of the element, and finally the original coordinates of the strand. Step 2516 also displays similar location information for wave-strand pairs that have the most and least separation for the model as a whole.

Although particular embodiments of the invention have been described in detail herein with reference to the accompanying drawings, it is to be understood that the invention is not limited to these precise embodiments, and that various changes and modifications may be effected therein by one skilled in the art without departing from the scope or spirit of the invention as defined by the appended claims.

What is claimed:

1. A method of steps to be performed on or by a computing device comprising a processor and memory, with said steps comprising:
receiving as input any one or more of dimensions, internal angles, orientation angles, rotation angles and rotation axis of a three-dimensional element with planar faces;
receiving as input a placement of a strand on or in said three dimensional element;
receiving as input parameters of a function that traces out said strand, wherein said function comprises one or more cosine or sine functions;
alteration of said a parameter of said function by a strain that arises from rotation of said three dimensional element;
tracking any one or more of inflection points, loops, paths, and extent of said strand.

2. The method of claim 1, wherein said strand represents any one or more of an atom, nucleon, and electron.

3. The method of claim 1, wherein said strand represents a wave.

4. A structure containing a model made from a method of steps, with the steps comprising:
receiving as input dimensions, internal angles, orientation angles, rotation angle and rotation axis of a three-dimensional element with planar faces;
receiving as input a placement of a strand on or in said three dimensional element;
receiving as input parameters of a function that traces out said strand, wherein said function comprises one or more cosine or sine functions;
alteration of a parameter of said function by a strain that arises from rotation of said three dimensional element;
tracking any one or more of inflection points, loops, paths, and extents of said strand.

5. The structure of claim 2, wherein said structure is comprised of one or more organic molecules.

6. The structure of claim 2, wherein said structure is comprised of any one or more of an atomic cluster, a portion of a lattice, and a grain of an inorganic material.

7. A mapping from a set of data to a set of information, with the mapping comprising a mapping of any of a set of data onto any one or more of dimensions, internal angles, orientation angles, rotation angle and rotation axis of a three-dimensional element with planar faces;
a mapping of any of initial data onto a strand that is traced out by a function comprising one or more cosine or sine functions;
computation of rotational strain from rotation of said three dimensional element;
alteration of a parameter of said function by a rotational strain from rotation of said three dimensional element;
a set of information that arises from any one or more of inflection points, loops, paths, extents, and sampling increments of said strand.

8. The mapping of claim 7, wherein said initial data are any one or more of a torque, speed, and energy.

9. The mapping of claim 7, wherein any one or more of an atom, nucleon, electron, atomic cluster, portion of a lattice and material grain map onto said three dimensional element and said strand.
